(19) United States
(12) Patent Application Publication
(10) Pub. No.: US 2023/0322857 A1
(43) Pub. Date: Oct. 12, 2023

(54) RECOMBINANTLY ENGINEERED POLYPEPTIDES CAPABLE OF RECYCLING NONCANONICAL COFACTORS

(71) Applicant: The Regents of the University of California, Oakland, CA (US)

(72) Inventor: Han Li, Irvine, CA (US)

(21) Appl. No.: 18/297,593

(22) Filed: Apr. 7, 2023

Related U.S. Application Data
(60) Provisional application No. 63/329,329, filed on Apr. 8, 2022.

(51) Int. Cl. C07K 7/08; C12N 9/02

(52) U.S. Cl. CPC C07K 7/08; C12Y 108/01007; C12N 9/0051; C12Y 108/01009

(57) ABSTRACT

Provided herein are recombinantly engineered polypeptides capable of recycling noncanonical cofactors, and uses thereof. Further provided herein, is a universal growth selection process that can be used to identify said recombinantly engineered polypeptides.

### CROSS REFERENCE TO RELATED APPLICATIONS

This application claims priority to U.S. Provisional Application Ser. No. 63/329,329, filed Apr. 8, 2022, the disclosure of which is incorporated herein by reference in its entirety.

### STATEMENT OF GOVERNMENT SUPPORT

This invention was made with Government support under Grant No. 1847705, awarded by the National Science Foundation, and Grant Nos. DE-AR0001508, and DP2 GM137427 awarded by the National Institutes of Health. The Government has certain rights in the invention.

### TECHNICAL FIELD

Provided herein are recombinantly engineered polypeptides capable of recycling noncanonical cofactors, and uses thereof. Further provided herein, is a universal growth selection process that can be used to identify said recombinantly engineered polypeptides.

### INCORPORATION BY REFERENCE OF SEQUENCE LISTING

This application includes a sequence listing entitled, “00058-072001.xml” with a ST26 Sequence Listing production date of 2023 Apr. 5 and having 10,900 bytes of data, machine formatted on IBM-PC, MS-Windows operating system using WIPO Standard ST.26 formatting. The sequence listing is hereby incorporated by reference in its entirety for all purposes.

### BACKGROUND

Enzymes catalyze many chemistries that are unattainable through organic synthesis. They perform these reactions renewably, operate under ambient conditions, and generate low waste. The most prevalent applications of biocatalysis involves the regio- and stereo-selective synthesis of chemicals which is reliant on nicotinamide adenine dinucleotide (phosphate) (NAD(P)⁺)-dependent oxidoreductases. These enzymes require stoichiometric input of NAD(P)⁺ for product formation, which constitutes a major cost that limits economic scalability. Attempts to reduce input costs through cofactor regeneration pathways still do not decrease costs sufficiently. This motivates the exploration of simpler NAD⁺ mimetics, or noncanonical redox cofactors, which retain the catalytic moiety of the native redox cofactors, but they are regularly structurally simpler and easier to synthesize. However, a significant hurdle blocking the widespread use of these simpler noncanonical cofactors is the lack of efficient and diverse enzymes that can utilize them. Except for some flavoenzymes, most enzymes engineered to use noncanonical cofactors do so with catalytic activities too low for practical applications.

### SUMMARY

Noncanonical redox cofactors are attractive low-cost alternatives to nicotinamide adenine dinucleotide (phosphate) (NAD(P)⁺) in biotransformation. However, engineering enzymes to utilize them is challenging. Disclosed herein is a high-throughput directed evolution platform which couples cell growth to the in vivo cycling of a noncanonical cofactor (e.g., nicotinamide mononucleotide (NMN⁺)). This is achieved by engineering the life-essential glutathione reductase in Escherichia coli to exclusively rely on the reduced NMN⁺ (NMNH). Using this growth selection system, a phosphite dehydrogenase (PTDH) was found to cycle NMN⁺ with ˜147-fold improved catalytic efficiency, which translates to an industrially viable total turnover number of ˜45,000 in a cell-free biotransformation without requiring high cofactor concentrations. Moreover, the PTDH variants also exhibited improved activity with another structurally deviant noncanonical cofactor, 1-benzylnicotinamide (BNA⁺), showcasing their broad applications. Importantly, structural modeling prediction reveals a general design principle where the mutations and the smaller, noncanonical cofactors together mimic the steric interactions of the larger, natural cofactors NAD(P)⁺.

In a particular embodiment, the disclosure provides a recombinantly engineered polypeptide that has improved catalytic efficiency for an oxidized form of a noncanonical cofactor, wherein the recombinantly engineered polypeptide comprises from 1 to 12 amino acid mutations in comparison to the sequence of the wild-type or parent polypeptide, or a thermostable variant thereof; wherein the amino acid mutations promote catalytic efficiency of the recombinantly engineered polypeptide in reducing the oxidized form of the noncanonical cofactor and/or disrupt electrostatic complementarity between the recombinantly engineered polypeptide and a natural cofactor, and wherein a polypeptide having the sequence of the wild-type or parent polypeptide, or a thermostable variant thereof, has minimal to very limited catalytic efficiency for the oxidized form of the noncanonical cofactor; wherein the recombinantly engineered polypeptide is configured to in vivo recycle the nonconical cofactor when used with a second recombinantly engineered polypeptide that utilizes the reduced form of noncanonical cofactor for cell growth. In a further embodiment, the recombinantly engineered polypeptide has at least 100-fold catalytic efficiency for the oxidized form of the noncanonical cofactor in comparison to the wild-type or parent polypeptide, or a thermostable variant thereof. In yet a further embodiment, the noncanonical cofactor is selected from the group consisting of nicotinamide mononucleotide (NMN), 1-phenyl-1,4,-dihydronicotinamide (PNA), 1-benzyl-1,4-dihydronicotinamide (BNA), 1-(4-hydroxyphenyl)1,4-dihydronicotinamide (HPNA), 1-methyl-1,4-dihydronicotinamide (MNA), nicotinamide flucytosine dinucleotide (NFCD), nicotinamide mononucleoside (NR), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile. In yet a further embodiment, the wild-type or parent polypeptide, or a thermostable variant thereof encodes a dehydrogenase or an oxidoreductase selected from phosphite dehydrogenase (PTDH), alcohol dehydrogenase (NAD), alcohol dehydrogenase (NADP), glutathione reductase, homoserine dehydrogenase, glucose dehydrogenase, glycerol dehydrogenase, propanediol-phosphate dehydrogenase, glycerol-3-phosphate dehydrogenase (NAD⁺), lactate dehydrogenase, malate dehydrogenase, isocitrate dehydrogenase, acetaldehyde dehydrogenase, glyceraldehyde 3-phosphate dehydrogenase, pyruvate dehydrogenase, oxoglutarate dehydrogenase, and formate dehydrogenase, or a thermostable variant of any one of the foregoing. In another embodiment, the recombinantly engineered polypeptide comprises a sequence that is at least 98% identical to the sequence of SEQ ID NO:3, SEQ ID NO:4, SEQ ID NO:5, or SEQ ID NO:6, and has PTDH activity.

In a certain embodiment, the disclosure also provides a recombinantly engineered polypeptide that has improved catalytic efficiency for a reduced form of a noncanonical cofactor, wherein the recombinantly engineered polypeptide comprises from 3 to 12 amino acid mutations in comparison to the sequence of the wild-type or parent polypeptide, or a thermostable variant thereof; wherein the amino acid mutations promote catalytic efficiency of the recombinantly engineered polypeptide for the reduced form of the noncanonical cofactor and/or reduces catalytic activity for natural cofactor(s), and wherein a polypeptide having the sequence of the wild-type or parent polypeptide, or a thermostable variant thereof, has minimal to very limited catalytic efficiency for the reduced form of the noncanonical cofactor; wherein the recombinantly engineered polypeptide uses the reduced noncanonical cofactor for cell growth. In a further embodiment, the recombinantly engineered polypeptide has at least 4-fold catalytic efficiency for the reduced form of the noncanonical cofactor in comparison to the wild-type or parent polypeptide, or a thermostable variant thereof. In yet a further embodiment, the noncanonical cofactor is selected from the group consisting of nicotinamide mononucleotide (NMN), 1-phenyl-1,4,-dihydronicotinamide (PNA), 1-benzyl-1,4-dihydronicotinamide (BNA), 1-(4-hydroxyphenyl)1,4-dihydronicotinamide (HPNA), 1-methyl-1,4-dihydronicotinamide (MNA), nicotinamide flucytosine dinucleotide (NFCD), nicotinamide mononucleoside (NR), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile. In another embodiment, the wild-type or parent polypeptide, or a thermostable variant thereof, has the sequence of SEQ ID NO:7. In yet another embodiment, the recombinantly engineered polypeptide comprises a sequence that is at least 98% identical to the sequence of SEQ ID NO:8, and has glutathione reductase activity.

In a particular embodiment, the disclosure provides a growth selection process to identify recombinantly engineered polypeptides capable of cycling an oxidized form of a noncanonical cofactor, the growth selection process comprising: expressing in microorganisms: (i) a first recombinantly engineered polypeptide that comprises from 3 to 12 amino acid mutations in comparison to the sequence of the wild-type or parent polypeptide, or thermostable variant thereof and which can utilize a reduced form of a noncanonical cofactor to promote microorganism growth; and (ii) a second recombinantly engineered polypeptide that comprises from 1 to 12 amino acid mutations in comparison to the sequence of the wild-type or parent polypeptide, or thermostable variant thereof, wherein the introduced amino acid mutations are designed to increase the activity of the recombinantly engineered polypeptide to reduce an oxidized form of the noncanonical cofactor; culturing the transformed microorganisms using a first selection criteria to identify variants with enhanced noncanonical-dependent activity, wherein the first selection criteria select for variants that show enhanced noncanonical cofactor activity by exhibiting an increased growth or growth rate in comparison to other transformed microorganisms; optionally, culturing the variants using additional selection criteria, wherein the additional selection criteria make it more, and more, challenging for the microorganisms to grow without having catalytic efficiency for the noncanonical cofactor. In a further embodiment the microorganisms are engineered to have an intracellular environment that is largely oxidative, by the disruption of gene(s) that encode protein(s) which reduce intracellular oxidative stress. In yet a further embodiment, the gene(s) for glutathione reductase (Gor) and/or thioredoxin reductase (TrxB) are disrupted. In another embodiment, the first recombinantly engineered polypeptide comprises a sequence that is at least 98% identical to the sequence of SEQ ID NO:8, and has glutathione reductase activity. In yet another embodiment, the second recombinantly engineered polypeptide comprises a sequence that is at least 85% identical to a sequence from a dehydrogenase or from an oxidoreductase. In a further embodiment, the dehydrogenase or the oxidoreductase is selected from phosphite dehydrogenase (PTDH), alcohol dehydrogenase (NAD), alcohol dehydrogenase (NADP), glutathione reductase, homoserine dehydrogenase, glucose dehydrogenase, glycerol dehydrogenase, propanediol-phosphate dehydrogenase, glycerol-3-phosphate dehydrogenase (NAD⁺), lactate dehydrogenase, malate dehydrogenase, isocitrate dehydrogenase, acetaldehyde dehydrogenase, glyceraldehyde 3-phosphate dehydrogenase, pyruvate dehydrogenase, oxoglutarate dehydrogenase, phosphite and formate dehydrogenase. In yet a further embodiment, the noncanonical cofactor is selected from the group consisting of nicotinamide mononucleotide (NMN), 1-phenyl-1,4,-dihydronicotinamide (PNA), 1-benzyl-1,4-dihydronicotinamide (BNA), 1-(4-hydroxyphenyl)1,4-dihydronicotinamide (HPNA), 1-methyl-1,4-dihydronicotinamide (MNA), nicotinamide flucytosine dinucleotide (NFCD), nicotinamide mononucleoside (NR), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile. In another embodiment, the amino acid mutations are selected by using a protein modeling program that predicts a protein structure de novo. In yet another embodiment, the first selection criteria include providing culture conditions comprising the noncanonical cofactor and a thiol oxidizing agent, and selecting for variants that exhibit the greatest growth and/or growth rates. In a further embodiment, the additional selection criteria include providing culture conditions of the first selection criteria but with a reduced concentration of a feedstock, and selecting for variants that exhibit the greatest growth and/or growth rates.

In a particular embodiment, the disclosure provides a recombinantly engineered polypeptide that has improved catalytic efficiency for an oxidized form of a noncanonical cofactor, wherein the recombinantly engineered polypeptide comprises from 1 to 12 amino acid mutations in comparison to the sequence of the wild-type or parent polypeptide, or a thermostable variant thereof wherein the amino acid mutations promote catalytic efficiency of the recombinantly engineered polypeptide in reducing the oxidized form of the noncanonical cofactor, and wherein a polypeptide having the sequence of the wild-type or parent polypeptide, or a thermostable variant thereof has minimal to very limited catalytic efficiency for the oxidized form of the noncanonical cofactor; wherein the recombinantly engineered polypeptide is configured to in vivo recycle the nonconical cofactor when used with a second recombinantly engineered polypeptide that utilizes the reduced form of noncanonical cofactor for cell growth. In a further embodiment, the recombinantly engineered polypeptide comprises 3 to 8 amino acid mutations in comparison to the sequence of the wild-type or parent polypeptide, or a thermostable variant thereof. In yet a further embodiment, the recombinantly engineered polypeptide has at least 100-fold catalytic efficiency for the oxidized form of the noncanonical cofactor in comparison to the wild-type or parent polypeptide, or a thermostable variant thereof. In another embodiment, the recombinantly engineered polypeptide further comprises from 1 to 8 additional amino acid mutations in comparison to the wild-type or parent polypeptide, or a thermostable variant thereof, wherein the additional amino acid mutations disrupt electrostatic complementarity between the recombinantly engineered polypeptide and a natural cofactor. In yet another embodiment, the additional amino acid mutations disrupt hydrogen bond formation between the recombinantly engineered polypeptide and a natural cofactor selected from flavin mononucleotide, flavin adenine dinucleotide, nicotinamide adenine dinucleotide, and nicotinamide adenine dinucleotide phosphate. In another embodiment, the noncanonical cofactor is selected from the group consisting of nicotinamide mononucleotide (NMN), 1-phenyl-1,4,-dihydronicotinamide (PNA), 1-benzyl-1,4-dihydronicotinamide (BNA), 1-(4-hydroxyphenyl)1,4-dihydronicotinamide (HPNA), 1-methyl-1,4-dihydronicotinamide (MNA), nicotinamide flucytosine dinucleotide (NFCD), nicotinamide mononucleoside (NR), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile. In a certain embodiment, the noncanonical cofactor is NMN or BNA. In a further embodiment, the recombinantly engineered polypeptide comprises a sequence that is at least 85% identical to a sequence from a dehydrogenase or an oxidoreductase. In yet a further embodiment, the dehydrogenase or the oxidoreductase is selected from phosphite dehydrogenase (PTDH), alcohol dehydrogenase (NAD), alcohol dehydrogenase (NADP), glutathione reductase, homoserine dehydrogenase, glucose dehydrogenase, glycerol dehydrogenase, propanediol-phosphate dehydrogenase, glycerol-3-phosphate dehydrogenase (NAD⁺), lactate dehydrogenase, malate dehydrogenase, isocitrate dehydrogenase, acetaldehyde dehydrogenase, glyceraldehyde 3-phosphate dehydrogenase, pyruvate dehydrogenase, oxoglutarate dehydrogenase, phosphite and formate dehydrogenase. In another embodiment, the dehydrogenase is a PTDH, and wherein the PTDH comprises sequence that is at least 98% identical to the sequence of SEQ ID NO:3, SEQ ID NO:4, SEQ ID NO:5, or SEQ ID NO:6.

In a particular embodiment, the disclosure also provides a growth selection process to identify recombinantly engineered polypeptides capable of cycling an oxidized form of a noncanonical cofactor, the growth selection process comprising: expressing in microorganisms: (i) a first recombinantly engineered polypeptide that can utilize a reduced form of a noncanonical cofactor to promote microorganism growth; and (ii) a second recombinantly engineered polypeptide that comprises from 1 to 12 amino acid mutations in comparison to the sequence of the wild-type or parent polypeptide, or a thermostable variant thereof, wherein the introduced amino acid mutations are designed to increase the activity of the recombinantly engineered polypeptide to reduce an oxidized form of the noncanonical cofactor; culturing the transformed microorganisms using a first selection criteria to identify variants with enhanced noncanonical-dependent activity, wherein the first selection criteria select for variants that show enhanced noncanonical cofactor activity by exhibiting an increased growth or growth rate in comparison to other transformed microorganisms; optionally, culturing the variants using additional selection criteria, wherein the additional selection criteria make it more, and more, challenging for the microorganisms to grow without having catalytic efficiency for the noncanonical cofactor. In another embodiment, the microorganisms are engineered to have an intracellular environment that is largely oxidative, by the disruption of gene(s) that encode protein(s) which reduce intracellular oxidative stress. In a certain embodiment, the gene(s) for glutathione reductase (Gor) and/or thioredoxin reductase (TrxB) are disrupted. In yet another embodiment, the first recombinantly engineered polypeptide comprises a sequence that is at least 98% identical to the sequence of SEQ ID NO:8, and has glutathione reductase activity. In a further embodiment, the second recombinantly engineered polypeptide comprises a sequence that is at least 85% identical to a sequence from a dehydrogenase or from an oxidoreductase. In yet a further embodiment, the dehydrogenase or the oxidoreductase is selected from phosphite dehydrogenase (PTDH), alcohol dehydrogenase (NAD), alcohol dehydrogenase (NADP), glutathione reductase, homoserine dehydrogenase, glucose dehydrogenase, glycerol dehydrogenase, propanediol-phosphate dehydrogenase, glycerol-3-phosphate dehydrogenase (NAD⁺), lactate dehydrogenase, malate dehydrogenase, isocitrate dehydrogenase, acetaldehyde dehydrogenase, glyceraldehyde 3-phosphate dehydrogenase, pyruvate dehydrogenase, oxoglutarate dehydrogenase, phosphite and formate dehydrogenase. In a further embodiment, the noncanonical cofactor is selected from the group consisting of nicotinamide mononucleotide (NMN), 1-phenyl-1,4,-dihydronicotinamide (PNA), 1-benzyl-1,4-dihydronicotinamide (BNA), 1-(4-hydroxyphenyl)1,4-dihydronicotinamide (HPNA), 1-methyl-1,4-dihydronicotinamide (MNA), nicotinamide flucytosine dinucleotide (NFCD), nicotinamide mononucleoside (NR), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile. In a certain embodiment, the noncanonical cofactor is NMN or BNA. In a further embodiment, the amino acid mutations are selected by using a protein modeling program that predicts a protein structure de novo. Examples of such protein modeling programs include, but are not limited to, trRosetta, Robetta, Rosetta@home, Abalone, or C-Quark. In a further embodiment, the first selection criteria include providing culture conditions comprising the noncanonical cofactor and a thiol oxidizing agent, and selecting for variants that exhibit the greatest growth and/or growth rates. In yet a further embodiment, wherein the additional selection criteria include providing culture conditions of the first selection criteria but with a reduced concentration of the feedstock, and selecting for variants that exhibit the greatest growth and/or growth rates.

In a particular embodiment, the disclosure further provides an recombinantly engineered polypeptide that is capable of recycling a noncanonical cofactor, wherein the recombinantly engineered polypeptide comprises 1, 2, 3, 4, 5, 6, 7, 8, 9, 10, 11, or 12 introduced amino acid mutations in comparison to the sequence of the wild-type or parent polypeptide, or a thermostable variant thereof; wherein the introduced amino acid mutations promote recycling of a noncanonical cofactor; wherein at least a portion of the introduced amino acid mutations were identified using a growth selection process disclosed herein; and wherein the noncanonical cofactor is a cofactor that is not normally utilized by the wild-type or parent polypeptide, or a thermostable variant thereof, and can be found in both a reduced or an oxidized form. In another embodiment, the recombinantly engineered polypeptide comprises 3, 4, 5, 6, 7 or 8 introduced amino acid mutations in comparison to the sequence of the wild-type or parent polypeptide, or a thermostable variant thereof. In yet another embodiment, the introduced amino acid substitution(s) for the recombinantly engineered polypeptide increase the catalytic efficiency for the noncanonical cofactor in comparison to the wild-type or parent polypeptide, or a thermostable variant thereof. In a further embodiment, the recombinantly engineered polypeptide has at least 10-fold catalytic efficiency for the noncanonical cofactor in comparison to the wild-type or parent polypeptide, or a thermostable variant thereof. In yet a further embodiment, the recombinantly engineered polypeptide has at least 100-fold catalytic efficiency for the noncanonical cofactor in comparison to the wild-type or parent polypeptide, or a thermostable variant thereof. In another embodiment, the recombinantly engineered polypeptide has at least 1000-fold catalytic efficiency for the noncanonical cofactor in comparison to the wild-type or parent polypeptide, or a thermostable variant thereof. In yet another embodiment, the recombinantly engineered polypeptide has greater specificity for the reduced form of the noncanonical cofactor v. the oxidized form of the noncanonical cofactor. In a further embodiment, the recombinantly engineered polypeptide has greater specificity for the oxidized form of the noncanonical cofactor v. the reduced form of the noncanonical cofactor. In yet a further embodiment,? the recombinantly engineered polypeptide further comprises 1, 2, 3, 4, 5, 6, 7, or 8 additional amino acid mutations in comparison to the wild-type or parent polypeptide, or a thermostable variant thereof, wherein the additional amino acid mutations disrupt electrostatic complementarity between the recombinantly engineered polypeptide and a natural cofactor. In a certain embodiment, the additional amino acid substitution(s) disrupt hydrogen bond formation between the recombinantly engineered polypeptide and a natural cofactor selected from flavin mononucleotide, flavin adenine dinucleotide, nicotinamide adenine dinucleotide, and nicotinamide adenine dinucleotide phosphate. In a further embodiment, the additional amino acid substitution(s) disrupt hydrogen bond formation between the recombinantly engineered polypeptide and nicotinamide adenine dinucleotide. In yet a further embodiment, the noncanonical cofactor is selected from the group consisting of nicotinamide mononucleotide (NMN), 1-phenyl-1,4,-dihydronicotinamide (PNA), 1-benzyl-1,4-dihydronicotinamide (BNA), 1-(4-hydroxyphenyl)1,4-dihydronicotinamide (HPNA), 1-methyl-1,4-dihydronicotinamide (MNA), nicotinamide flucytosine dinucleotide (NFCD), nicotinamide mononucleoside (NR), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile. In a particular embodiment, the noncanonical cofactor is NMN or BNA. In a further embodiment, the recombinantly engineered polypeptide has a decrease of 30-fold or more in catalytic activity for the natural cofactor in comparison to the wild-type or parent polypeptide, or a thermostable variant thereof. In yet a further embodiment, the recombinantly engineered polypeptide comprises a sequence that is at least 85% identical to a sequence from a dehydrogenase or an oxidoreductase. In another embodiment, the dehydrogenase or the oxidoreductase is selected from phosphite dehydrogenase (PTDH), alcohol dehydrogenase (NAD), alcohol dehydrogenase (NADP), glutathione reductase, homoserine dehydrogenase, glucose dehydrogenase, glycerol dehydrogenase, propanediol-phosphate dehydrogenase, glycerol-3-phosphate dehydrogenase (NAD⁺), lactate dehydrogenase, malate dehydrogenase, isocitrate dehydrogenase, acetaldehyde dehydrogenase, glyceraldehyde 3-phosphate dehydrogenase, pyruvate dehydrogenase, oxoglutarate dehydrogenase, phosphite and formate dehydrogenase. In yet another embodiment, the dehydrogenase is a PTDH. In a further embodiment, the PTDH comprises sequence that is at least 98% identical to the sequence of SEQ ID NO:3, SEQ ID NO:4, SEQ ID NO:5, or SEQ ID NO:6. In yet a further embodiment, the oxidoreductase is a glutathione reductase. In another embodiment, the glutathione reductase comprises sequence that is at least 98% identical to the sequence of SEQ ID NO:8.

In a particular embodiment, the disclosure also provides a growth selection process to identify recombinantly engineered polypeptides capable of using a noncanonical cofactor, the growth selection process comprising: expressing in microorganisms: (i) a first recombinantly engineered polypeptide that can utilize a reduced form of a noncanonical cofactor for generating a reduced compound essential for bacterial survival and growth; and (ii) recombinantly engineered polypeptide(s) that comprise 1, 2, 3, 4, 5, 6, 7, 8, 9, 10, 11, or 12 introduced amino acid mutations in comparison to the sequence of the wild-type or parent polypeptide, or a thermostable variant thereof, wherein the introduced amino acid mutations are designed to increase the activity of the recombinantly engineered polypeptide(s) for an oxidized form of the noncanonical cofactor; culturing the transformed microorganisms using a first selection criteria to identify variants with enhanced noncanonical-dependent activity, wherein the first selection criteria select for microorganisms that show enhanced noncanonical cofactor activity by exhibiting an increased growth or growth rate in comparison to other microorganisms; optionally, culturing the variants using additional selection criteria, wherein the additional selection criteria making it more, and more, challenging for the microorganisms to grow without having noncanonical cofactor activity. In a further embodiment, the first recombinantly engineered polypeptide comprises a sequence that is at least 98% identical to the sequence of SEQ ID NO:8. In yet a further embodiment, the introduced amino acid mutations were selected by using a protein modeling program. In another embodiment, the recombinantly engineered polypeptide(s) comprises a sequence that is at least 85% identical to a sequence from a dehydrogenase or an oxidoreductase. In a further embodiment, the dehydrogenase or the oxidoreductase is selected from phosphite dehydrogenase (PTDH), alcohol dehydrogenase (NAD), alcohol dehydrogenase (NADP), glutathione reductase, homoserine dehydrogenase, glucose dehydrogenase, glycerol dehydrogenase, propanediol-phosphate dehydrogenase, glycerol-3-phosphate dehydrogenase (NAD⁺), lactate dehydrogenase, malate dehydrogenase, isocitrate dehydrogenase, acetaldehyde dehydrogenase, glyceraldehyde 3-phosphate dehydrogenase, pyruvate dehydrogenase, oxoglutarate dehydrogenase, phosphite and formate dehydrogenase. In yet a further embodiment, the noncanonical cofactor is selected from the group consisting of nicotinamide mononucleotide (NMN), 1-phenyl-1,4,-dihydronicotinamide (PNA), 1-benzyl-1,4-dihydronicotinamide (BNA), 1-(4-hydroxyphenyl)1,4-dihydronicotinamide (HPNA), 1-methyl-1,4-dihydronicotinamide (MNA), nicotinamide flucytosine dinucleotide (NFCD), nicotinamide mononucleoside (NR), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile. In another embodiment, the noncanonical cofactor is NMN or BNA. In yet another embodiment, the first selection criteria include providing culture conditions comprising the noncanonical cofactor, and selecting for variants that showed enhanced noncanonical cofactor dependent activity. In a certain embodiment, the additional selection criteria include providing more challenging culture conditions that comprise the noncanonical cofactor, and a limited supply of a feedstock.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the disclosure and, together with the detailed description, serve to explain the principles and implementations of the invention.

FIG. 1A-E provides (A) Schematic of NMN(H) redox system coupling NMN⁺ reducing enzyme and life-essential glutathione reductase in an engineered E. coli strain. Redox imbalance through low supply of NMNH results in insufficient GSH production and protein misfolding. (B) Structure of the coenzyme binding pocket in Gor from E. coli (PDB: 1GET). Residues interacting with the cofactors are shown as cyan sticks, the native cofactor NADPH is colored green, and the FAD is colored yellow. (C) In vitro NMNH-dependent activity of eight single-mutation variants in the first round of rational design. (D) Kinetic characterization of Gor variants. Activities were measured as described in Methods. (E) Comparison of growth behavior for WT and E. coli SHuffle strain with coupled or uncoupled coenzyme recycling system at varying NMN⁺ concentrations. SHuffle with wild type Gor experiences no growth defects, while SHuffle with Gor Ortho only grows with NMNH regeneration. Values represent the average of two or three biological replicates. Error bars represent one standard deviation. Two-tailed t-tests were used to determine statistical significance (*P<0.05).

FIG. 2 presents a model of NMN⁺ binding pose with TS-PTDH I178T. The hydrophobic I178 is unable to make attractive polar contacts with NMN⁺. With mutation to I178T, the threonine is positioned to form hydrogen bonds with the NMN⁺ phosphate group.

FIG. 3A-C provides TS-PTDH active site bioinformatic analysis. (A) First-shell binding residues surrounding NMN₊ were identified as positions containing any atom within 6 Å of the predicted NMN⁺ binding pose. Since NMN⁺ and NAD⁺ share the same core structure with hydride transfer occurring at the nicotinamide ring C4, it was postulated that the active binding mode will be almost identical. Five regions were selected for conservation analysis: (1) the seatbelt loop from 76-79 named for the characteristic K76 found in many PTDHs that reaches across the binding pocket to form a polar contact with the pyrophosphates, (2) the arch above which includes 97, 100, 101, and 104, (3) the Rossmann alpha helix 1 from 154-157, (4) the base loop running from 207-210, and (5) the nicotinamide pocket which includes 235-237, 261-262, and 292-295. Numbered residues are illustrated in A. (B) Positional conservation of PTDH residues. The raw signal is illustrated as a medium gray line, and a smoothed signal with 6-window moving average is shown in light-medium gray line to focus on regional variation. The conservation score is a relative ranking with higher values indicating that the position has greater conservation, and lower values meaning greater variance. (C) Mean conservation scores for the regions composing the NMN⁺ binding site. Residues belonging to the specified region are represented as a light-medium gray dot, and the standard deviation of the conservation scores is shown as error bars, there are no replicates, as this is an aggregate of the individual positions. All regions show a high degree of conservation with values ranging from 50±4% for the base loop to 74±4% for the Rossmann alpha helix 1. Mutations in these areas would strongly affect NAD⁺ turnover due to their direct contact and variants with reduced catalytic efficiency for NAD⁺ would suffer a considerable loss in fitness. As usage of mNADs is not found naturally, there is little selection pressure to sample sequences away from those already proficient with NAD⁺. The bioinformatic analysis of the cofactor active site is ineffective in discovering evolvable, variable regions that readily tolerate mutations for mNAD engineering. Analysis was performed as described in the Examples section.

FIG. 4A-B shows TS-PTDH active site residue frequencies. (A) First shell residues enveloping the NMN+ (green lines) are colored according to their regions illustrated in FIG. 3A, each residue alpha carbon is represented as a sphere and labeled with the amino acid identity and position. (B) The distribution of residue frequencies with gaps ignored for each position was calculated from multiple sequence alignment (MSA) of TS-PTDH homologs found from BLAST-P search and colored by frequency with yellow indicating 0% observation to dark blue indicating 100% observation. The wild-type residue at each position is highlighted with red outline and text annotation. This analysis was performed to detect if any position was amenable to sampling polar residues that are arranged to potentially form favorable electrostatic contact with the NMN+ phosphate and to visualize the types of residues natively found. The existence of any position with recurrent substitution to polar residues would give strong evidence that the mutations would be tolerated on the TS-PTDH scaffold and additionally the sequences with the substitutions could be an alternative template for engineering. The results exhibit severe purifying selection as all regions are highly conserved and the small amount of variability observed is generally limited to positions swapping between small polar or non-polar amino acids. Due to the lack of suggested variants from evolutionary analysis, we focus on applying structure-based rational design to engineer NMN+ binding.

FIG. 5 presents a binding mode of the natural cofactor NAD⁺ in TS-PTDH (PDB: 4E5N). First shell residues lining the cofactor binding pocket are indicated by cyan sticks. Critical side-chain polar contacts that secure NAD⁺ include K76 which reaches from a loop across the binding interface to form salt bridges with the pyrophosphate, and E175 that establishes a bidentate hydrogen bond with the adenosine ribose.

FIG. 6A-C presents first round of TS-PTDH variants engineered through rational design. (A) Two mutants (green) displayed specific activities with NMN⁺ notably greater than the wild type (blue). A155N showed -3-fold increase and T101K-A155G showed a ˜2-fold increase compared to the wild type. (B) Molecular modeling of the designs suggests that T101K-A155G functions through the cooperative effects of A155G clearing space for T101K to project over to form a salt bridge with the NMN⁺ phosphate. (C) A155N forms a hydrogen bond with K76 that reinforces the existing salt bridge between K76 and the phosphate group. This interaction may support pre-organization of K76 to grasp the cofactor upon binding. Values represent the average of at least two biological replicates. Error bars represent one standard deviation. Two-tailed t-tests were used to determine statistical significance (*P<0.05).

FIG. 7 presents an analysis of library quality at site saturated mutagenesis targeted residues. Sanger sequencing of the TS-PTDH library used for growth selection shows a distribution of bases at each NNK site. Library quality was determined as described in Sullivan et al. (Enzyme Microb. Technol. 53:70-77 (2013). Briefly, peak heights of the fluorescent chromatogram from Sanger Sequencing were determined at each nucleotide position, summed, and standardized by the summed height at that base, as shown in the tables below each chromatogram. Relative height values were then used to determine the summed amount of deviation from the expected distribution, Σdev. For example, an “N” basepair should show an even 25% relative height of each of the four bases, where a “K” site should show only a 50% relative distribution between “T” and “G”. Finally, the Qₚₒₒₗ value is determined as a weighted metric of each base in the codon. Qₚₒₒₗ values >0.7 are regarded as satisfactory for library quality.

FIG. 8 demonstrates recapitulation of growth restoration phenotype for E. coli Shuffle strain with wild-type TS-PTDH, variants from rational design, and variants identified in selection spotted onto agar plates. Co-expression of wild type TS-PTDH and wild type Ec Gor supported cell growth, while cells with co-expression of wild type TS-PTDH and NMNH-specific Gor Ortho grew poorly. When TS-PTDH mutants were introduced, co-expression of TS-PTDH A155N with NMNH-specific Gor Ortho restored growth at a low level, but co-expression of selected TS-PTDH LY-13 (A155N-E175A-A176F) with NMNH-specific Gor Ortho enabled improved growth compared to TS-PTDH A155N. Co-expression of an empty plasmid and wild-type Gor struggled to grow in the same conditions.

FIG. 9A-D demonstrates structural modeling of PTDH variants discovered through growth selection with A155N template. (A) Cartoon of TS-PTDH depicting residue packing at the AMP binding pocket in LY-7. In TS-PTDH, NAD⁺ is docked in the binding pocket. In LY-7, NMN⁺ is docked in the binding pocket. (B) In LY-6 (A155N-E175Q-A176S), E175Q maintains similar steric properties of the original glutamate, but the change from the carboxylic acid to amide enables E175Q to act as a hydrogen bond donor that bridges separate loops in the adenine binding cleft for improved stability. The backbone amide of M153 contacts the oxygen on E175Q and the hydroxyl from A176S accepts a hydrogen bond from the E175Q nitrogen. (C) LY-7 (A155N-E175W-A176G-L208V) is characterized by dense hydrophobic packing in the adenine cleft. A176G allows greater loop flexibility and room for E175W to adopt a rotamer that occupies the open void where the adenine would typically fit. L208V results in a slightly smaller side chain with greater shape complementarity to pack against the bulky E175W. (D) LY-13 (A155N-E175A-A176F) displays similar hydrophobic packing in the adenine cleft as LY-7 with E175A shrinking and A176F settling against L208 and P209 in the adenine cleft.

FIG. 10A-D demonstrates applying TS-PTDH to reductive biotransformation. (A) redox cofactor cycling system was constructed using PTDH as a reduced cofactor regeneration source. (B) Reaction progression curve for determining the TTN of LY-13 in the redox cycling system employing NMN(H). A low concentration of LY-13, 0.25 μM, requires high protein turnover to enable the reaction to progress. (C) Comparison of using LY-13 or an NMNH-active glucose dehydrogenase (GDH Triple) as NMNH recycling enzymes. 10 μM LY-13 or GDH Triple were supplied to the cycling system containing varying concentrations of NMN⁺. The lower Kₘ of LY-13 enables more efficient utilization of the NMN⁺ pool at lower cofactor concentrations. (D) The synthetic noncanonical cofactor BNA⁺ was substituted for NMN⁺ in the cycling system. Despite the highly deviant structure of BNA⁺ compared with NMN⁺, the TS-PTDH variants were able to recycle BNA(H) at a higher rate than the wild type protein. Values represent the average of three biological replicates. Error bars represent one standard deviation.

FIG. 11 shows that the reversion of the A155 mutation in LY-13 retains activity with BNA⁺. The A155N mutation in LY-13, the highest performing TS-PTDH variant with BNA⁺, was reverted to an alanine. When LY-13 A155A was cycled with TsOYE and BNA⁺, biotransformation activity remained consistent with LY-13. Reactions were performed as described in the “BNA(H)-Mediated PTDH Biotransformation” methods section. Values represent the average of three biological replicated. Error bars represent one standard deviation. Statistical significance was determined by two-tailed t-test, (*P<0.05).

FIG. 12 presents the sequence of a wild type phosphite dehydrogenase from Pseudomonas stutzeri (SEQ ID NO:1).

FIG. 13 presents the sequence of a variant phosphite dehydrogenase from Pseudomonas stutzeri that has been mutated to be more thermostable (SEQ ID NO:2).

FIG. 14 presents the sequence of a recombinantly engineered polypeptide of a phosphite dehydrogenase from Pseudomonas stutzeri with an A155N substitution indicated in bold type (SEQ ID NO:3).

FIG. 15 presents the sequence of a recombinantly engineered polypeptide of a phosphite dehydrogenase from Pseudomonas stutzeri with the mutations from LY-6 (A155N, E175Q, A176S) indicated in bold type (SEQ ID NO:4).

FIG. 16 presents the sequence of a recombinantly engineered polypeptide of a phosphite dehydrogenase from Pseudomonas stutzeri with the mutations from LY-7 (A155N, E175W, A176G, L208V) indicated in bold type (SEQ ID NO:5).

FIG. 17 presents the sequence of a recombinantly engineered polypeptide of a phosphite dehydrogenase from Pseudomonas stutzeri with the mutations from LY-13 (A155N, E175A, A176F) indicated in bold type (SEQ ID NO: 6).

FIG. 18 presents the sequence of a wild type polypeptide of a glutathione reductase from Escherichia coli (SEQ ID NO:7).

FIG. 19 presents the sequence of a recombinantly engineered polypeptide of a glutathione reductase from Escherichia coli with the mutations from Gor Ortho (I178T, R198M, R204L) indicated in bold type (SEQ ID NO:8).

### DETAILED DESCRIPTION

As used herein and in the appended claims, the singular forms “a,” “an,” and “the” include plural referents unless the context clearly dictates otherwise. Thus, for example, reference to “a polynucleotide” includes a plurality of such polynucleotides and reference to “the oxidoreductase” includes reference to one or more oxidoreductases, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

All publications mentioned herein are incorporated by reference in full for the purpose of describing and disclosing methodologies that might be used in connection with the description herein. Moreover, with respect to any term that is presented in one or more publications that is similar to, or identical with, a term that has been expressly defined in this disclosure, the definition of the term as expressly provided in this disclosure will control in all respects.

As used herein, a “natural cofactor” refers to a non-protein chemical compound or metallic ion that is normally required for an enzyme's activity as a catalyst. Natural cofactors can be either loosely or tightly bound to the enzyme and can directly participate in the reaction. For example, NAD⁺ or NADP⁺ are natural cofactors for glucose dehydrogenase that are required for the enzyme's activity. In direct contrast, NMN⁺ is not a natural cofactor for glucose dehydrogenase, and therefore is not required for the enzyme's in vivo activity.

As used herein, an “noncanonical cofactor” refers to a chemical compound or metallic ion that is not normally required or associated with a particular enzyme's activity as a catalyst, but by the result of mutations or other changes, the enzyme's activity towards the noncanonical cofactor can be greatly enhanced. For example, NMN⁺ is not a natural cofactor for glucose dehydrogenase, but by introducing mutations into the polypeptide sequence for glucose dehydrogenase, the glucose dehydrogenase's activity towards NMN⁺ can be greatly enhanced.

As used herein, a “mutation” means any process or mechanism resulting in a mutant protein, enzyme, polynucleotide, gene, or cell. This includes any mutation in which a protein, enzyme, polynucleotide, or gene sequence is altered, and any detectable change in a cell arising from such a mutation. Typically, a mutation occurs in a polynucleotide or gene sequence, by point mutations, deletions, or insertions of single or multiple nucleotide residues. A mutation includes polynucleotide alterations arising within a protein-encoding region of a gene as well as alterations in regions outside of a protein-encoding sequence, such as, but not limited to, regulatory or promoter sequences. A mutation in a gene can be “silent”, i.e., not reflected in an amino acid alteration upon expression, leading to a “sequence-conservative” variant of the gene. This generally arises when one amino acid corresponds to more than one codon.

Non-limiting examples of a modified amino acid include a glycosylated amino acid, a sulfated amino acid, a prenlyated (e.g., farnesylated, geranylgeranylated) amino acid, an acetylated amino acid, an acylated amino acid, a pegylated amino acid, a biotinylated amino acid, a carboxylated amino acid, a phosphorylated amino acid, and the like. References adequate to guide one of skill in the modification of amino acids are replete throughout the literature. Example protocols are found in Walker (1998) Protein Protocols on CD-ROM (Humana Press, Towata, N.J.).

Recombinant methods for producing and isolating modified polypeptides of the disclosure are described herein. In addition to recombinant production, the polypeptides may be produced by direct peptide synthesis using solid-phase techniques (e.g., Stewart et al. (1969) Solid-Phase Peptide Synthesis (WH Freeman Co, San Francisco); and Merrifield (1963) J. Am. Chem. Soc. 85: 2149-2154). Peptide synthesis may be performed using manual techniques or by automation. Automated synthesis may be achieved, for example, using Applied Biosystems 431A Peptide Synthesizer (Perkin Elmer, Foster City, Calif.) in accordance with the instructions provided by the manufacturer.

A “reductase” refers to an enzyme that catalyzes the transfer of electrons from one molecule, the reductant, also called the electron donor, to another, the oxidant, also called the electron acceptor. A “oxidoreductase” refers to an enzyme allows an oxidation to occur as well as a reduction to occur. Examples of oxidoreductases and reductases include those enzymes that act on the CH—OH group of donors with NAD⁺ or NADP⁺ as an acceptor, including alcohol dehydrogenase (NAD) (EC 1.1.1.1), alcohol dehydrogenase (NADP) (EC 1.1.1.2), homoserine dehydrogenase (EC 1.1.1.3), aminopropanol oxidoreductase (EC 1.1.1.4), diacetyl reductase (EC 1.1.1.5), glycerol dehydrogenase (EC 1.1.1.6), propanediol-phosphate dehydrogenase (EC 1.1.1.7), glycerol-3-phosphate dehydrogenase (NAD⁺) (EC 1.1.1.8), D-xylulose reductase (EC 1.1.1.9), L-xylulose reductase (EC 1.1.1.10), lactate dehydrogenase (EC 1.1.1.27), malate dehydrogenase (EC 1.1.1.37), isocitrate dehydrogenase (EC 1.1.1.42), and HMG-CoA reductase EC (1.1.1.88); enzymes that act on the CH-OH group of donors with oxygen as an acceptor, including glucose oxidase (EC 1.1.3.4), L-gulonolactone oxidase (EC 1.1.3.8), thiamine oxidase (EC 1.1.3.23), xanthine oxidase (EC 1.1.3.32); enzymes that act on the aldehyde or oxo group of donors with NAD⁺ or NADP⁺ as an acceptor, including acetaldehyde dehydrogenase EC (1.2.1.10), glyceraldehyde 3-phosphate dehydrogenase (EC 1.2.1.12), pyruvate dehydrogenase (EC 1.2.1.51), oxoglutarate dehydrogenase (EC 1.2.4.2); enzymes that act on the CH—CH group of donors with NAD⁺ or NADP⁺ as an acceptor, including biliverdin reductase (EC 1.3.1.24); enzymes that act on CH—CH group of donors with oxygen as an acceptor, including protoporphyrinogen oxidase (EC 1.3.3.4); enzymes that act on the CH—NH₂ group of donors, including monoamine oxidase (EC 1.4.3.4); enzymes that act on the CH—NH group of donors with NAD⁺ or NADP⁺ as an acceptor, including dihydrofolate reductase (EC 1.5.1.3), and methylenetetrahydrofolate reductase (EC 1.5.1.20); enzymes that act on the CH—NH group of donors with oxygen as an acceptor, sarcosine oxidase (EC 1.5.3.1), and dihydrobenzophenanthridine oxidase (EC 1.5.3.12); enzymes that act on other nitrogenous compounds as donors, including urate oxidase (EC 1.7.3.3), nitrite reductase (EC 1.7.99.3), and nitrate reductase (EC 1.7.99.4); enzymes that act on the sulfur group of donors, including glutathione reductase (EC 1.8.1.7), thioredoxin reductase (EC 1.8.1.9), and sulfite oxidase (EC 1.8.3.1); enzymes that act on the heme group of donors, including cytochrome c oxidase (EC 1.9.3.1); enzymes that act on diphenols and related substances as donors, including coenzyme Q-cytochrome c reductase (EC 1.10.2.2), catechol oxidase (EC 1.10.3.1), and laccase (EC 1.10.3.2); enzymes that act on peroxide as acceptor, including Cytochrome c peroxidase (EC 1.11.1.5), catalase (EC 1.11.1.6), myeloperoxidase (EC 1.11.1.7), thyroid peroxidase (EC 1.11.1.8), and glutathione peroxidase (EC 1.11.1.9); enzymes that act on single donors with incorporation of molecular oxygen, 4-hydroxyphenylpyruvate dioxygenase (EC 1.13.11.27), renilla-luciferin 2-monooxygenase (EC 1.13.12.5), cypridina-luciferin 2-monooxygenase (EC 1.13.12.6), Firefly luciferase (EC 1.13.12.7), watasenia-luciferin 2-monooxygenase (EC 1.13.12.8), and oplophorus-luciferin 2-monooxygenase EC (1.13.12.13); enzymes that act on paired donors with incorporation of molecular oxygen, including aromatase (EC 1.14.14.1), CYP2D6 (EC 1.14.14.1), CYP2E1 (EC 1.14.14.1), CYP3A4 (EC 1.14.14.1), Cytochrome P450 oxidase, nitric oxide synthase (EC 1.14.13.39), phenylalanine hydroxylase (EC 1.14.16.1), and tyrosinase (EC 1.14.18.1); and other oxidoreductases, including superoxide dismutase (EC 1.15.1.1), nitrogenase (EC 1.18.6.1), and deiodinase (EC 1.97.1.10). The above listing, provides for the classification of the foregoing enzymes by in the International Union of Biochemistry and Molecular Biology's Enzyme Commission [EC] numbering system. In a particular embodiment, the disclosure provides for an recombinantly engineered polypeptide based upon an oxidoreductase disclosed above, that has been engineered to contain amino acid mutations so as to enable the efficient recycling of a noncanonical cofactor. In a further embodiment, the oxidoreductase is a glutathione reductase.

“Dehydrogenase” means an enzyme belonging to the group of oxidoreductases that oxidizes a substrate by reducing an electron acceptor, usually NAD⁺/NADP⁺ or a flavin coenzyme such as FAD or FMN. They also catalyze the reverse reaction, for instance alcohol dehydrogenase not only oxidizes ethanol to acetaldehyde in animals but also produces ethanol from acetaldehyde in yeast. In another embodiment, the disclosure provides for an recombinantly engineered polypeptide based upon a dehydrogenase, that has been engineered to contain amino acid mutations so as to enable the efficient recycling of an noncanonical cofactor.

A “protein” or “polypeptide”, which terms are used interchangeably herein, comprises one or more chains of chemical building blocks called amino acids that are linked together by chemical bonds called peptide bonds. An “enzyme” means any substance, preferably composed wholly or largely of protein, that catalyzes or promotes, more or less specifically, one or more chemical or biochemical reactions. A “native” or “wild-type” protein, enzyme, polynucleotide, gene, or cell, means a protein, enzyme, polynucleotide, gene, or cell that occurs in nature.

An “amino acid sequence” is a polymer of amino acids (a protein, polypeptide, etc.) or a character string representing an amino acid polymer, depending on context. The terms “protein,” “polypeptide,” and “peptide” are used interchangeably herein. “Amino acid” is a molecule having the structure wherein a central carbon atom is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino acid carboxylic groups in the dehydration reaction that links one amino acid to another. As a result, when incorporated into a protein, an amino acid is referred to as an “amino acid residue.”

A particular amino acid sequence of a given protein (i.e., the polypeptide's “primary structure,” when written from the amino-terminus to carboxy-terminus) is determined by the nucleotide sequence of the coding portion of a mRNA, which is in turn specified by genetic information, typically genomic DNA (including organelle DNA, e.g., mitochondrial or chloroplast DNA). Thus, determining the sequence of a gene assists in predicting the primary sequence of a corresponding polypeptide and more particular the role or activity of the polypeptide or proteins encoded by that gene or polynucleotide sequence.

“Conservative amino acid substitution” or, simply, “conservative substitution” of a particular sequence refers to the replacement of one amino acid, or series of amino acids, with essentially identical amino acid sequences. One of skill will recognize that individual mutations, deletions or additions which alter, add or delete a single amino acid or a percentage of amino acids in an encoded sequence result in “conservative variations” where the alterations result in the deletion of an amino acid, addition of an amino acid, or substitution of an amino acid with a chemically similar amino acid. For purposes of this disclosure a “conservative amino acid substitution” does significantly affect the catalytic activity towards an noncanonical cofactor and/or structural stability of an recombinantly engineered polypeptide disclosed herein. For example, the recombinantly engineered polypeptide of the disclosure may comprise conservative amino acid mutations in regions of the sequence that do not impact the binding site for the noncanonical cofactor, e.g., conservative amino acid changes on the surface of the protein. Further, the sequence of an recombinantly engineered polypeptide disclosed herein can be aligned with polypeptide sequence(s) from enzymes that have similar structures and/or catalytic activity in order to identify amino acids that likely do not affect the catalytic activity and/or structural stability of the recombinantly engineered polypeptide. Moreover, there are many protein modeling programs available, including those specifically recited herein (e.g., Spartan and RosettaDesign), which can identify conservative amino acid mutations with a high degree of probability/certainty that would not significantly affect the catalytic activity and/or structural stability of an recombinantly engineered polypeptide disclosed herein (e.g., see Ng et al., Predicting Deleterious Amino Acid Changes Genome Res 11:863-874 (2001)). As such, it is expected that one of skill in the art could reasonably predict that the sequence for an recombinantly engineered polypeptide disclosed herein can comprise a percentage of conservative amino acid mutations, as is described more fully below, and still have similar or the same catalytic activity for the noncanonical cofactor as a polypeptide sequence specifically recited herein (e.g., SEQ ID NO:6 or SEQ ID NO:8). Similar reasoning applies for the structural stability of an recombinantly engineered polypeptide disclosed herein.

Conservative substitution tables providing functionally similar amino acids are well known in the art. For example, one conservative substitution group includes Alanine (A), Serine (S), and Threonine (T). Another conservative substitution group includes Aspartic acid (D) and Glutamic acid (E). Another conservative substitution group includes Asparagine (N) and Glutamine (Q). Yet another conservative substitution group includes Arginine (R) and Lysine (K). Another conservative substitution group includes Isoleucine, (I) Leucine (L), Methionine (M), and Valine (V). Another conservative substitution group includes Phenylalanine (F), Tyrosine (Y), and Tryptophan (W).

Thus, “conservative amino acid mutations” of a polypeptide sequence disclosed herein include mutations of a percentage, typically less than 5%, 6%, 7%, 8%, 9%, or 10%, of the amino acids of the polypeptide sequence, with a conservatively selected amino acid of the same conservative substitution group. Accordingly, a conservatively substituted variation of a polypeptide of the disclosure can contain 1, 2, 3, 4, 5, 6, 7, 8, 9, 10, 15, 20, 25, or any range that includes or is in between mutations with a conservatively substituted variation of the same conservative substitution group.

It is understood that the addition of sequences which do not alter the encoded activity of a nucleic acid molecule, such as the addition of a non-functional or non-coding sequence, is a conservative variation of the basic nucleic acid. The “activity” of an enzyme is a measure of its ability to catalyze a reaction, i.e., to “function”, and may be expressed as the rate at which the product of the reaction is produced. For example, enzyme activity can be represented as the amount of product produced per unit of time or per unit of enzyme (e.g., catalytic efficiency), or in terms of affinity or dissociation constants.

One of skill in the art will appreciate that many conservative variations of the nucleic acid constructs which are disclosed yield a functionally identical construct. For example, as discussed above, owing to the degeneracy of the genetic code, “silent mutations” (i.e., mutations in a nucleic acid sequence which do not result in an alteration in an encoded polypeptide) are an implied feature of every nucleic acid sequence which encodes an amino acid. Similarly, “conservative amino acid mutations,” in one or a few amino acids in an amino acid sequence are substituted with different amino acids with highly similar properties, are also readily identified as being highly similar to a disclosed construct. Such conservative variations of each disclosed sequence are a feature of the polypeptides provided herein.

“Conservative variants” are proteins or enzymes in which a given amino acid residue has been changed without altering overall conformation and function of the protein or enzyme, including, but not limited to, replacement of an amino acid with one having similar properties, including polar or non-polar character, size, shape and charge. Amino acids other than those indicated as conserved may differ in a protein or enzyme so that the percent protein or amino acid sequence similarity (or identity) between any two proteins of similar function may vary and can be, for example, at least 30%, at least 50%, at least 70%, at least 80%, or at least 90%, as determined according to an alignment scheme. As referred to herein, “sequence similarity” means the extent to which nucleotide or protein sequences are related. The extent of similarity between two sequences can be based on percent sequence identity and/or conservation. “Sequence identity” herein means the extent to which two nucleotide or amino acid sequences are invariant. “Sequence alignment” means the process of lining up two or more sequences to achieve maximal levels of identity (and, in the case of amino acid sequences, conservation) for the purpose of assessing the degree of similarity. Numerous methods for aligning sequences and assessing similarity/identity are known in the art such as, for example, the Cluster Method, wherein similarity is based on the MEGALIGN algorithm, as well as BLASTN, BLASTP, and FASTA (Lipman and Pearson, 1985; Pearson and Lipman, 1988). When using all of these programs, the preferred settings are those that results in the highest sequence similarity.

Non-conservative modifications of a particular polypeptide are those which substitute any amino acid not characterized as a conservative substitution. For example, any substitution which crosses the bounds of the six groups set forth above. These include mutations of basic or acidic amino acids for neutral amino acids, (e.g., Asp, Glu, Asn, or Gln for Val, Ile, Leu or Met), aromatic amino acid for basic or acidic amino acids (e.g., Phe, Tyr or Trp for Asp, Asn, Glu or Gln) or any other substitution not replacing an amino acid with a like amino acid. Basic side chains include lysine (K), arginine (R), histidine (H); acidic side chains include aspartic acid (D), glutamic acid (E); uncharged polar side chains include glycine (G), asparagine(N), glutamine (Q), serine (S), threonine (T), tyrosine (Y), cysteine (C); nonpolar side chains include alanine (A), valine (V), leucine (L), isoleucine (I), proline (P), phenylalanine (F), methionine (M), tryptophan (W); beta-branched side chains include threonine (T), valine (V), isoleucine (I); aromatic side chains include tyrosine (Y), phenylalanine (F), tryptophan (W), and histidine (H).

A “parent” protein, enzyme, polynucleotide, gene, or cell, is any protein, enzyme, polynucleotide, gene, or cell, from which any other protein, enzyme, polynucleotide, gene, or cell, is derived or made, using any methods, tools or techniques, and whether or not the parent is itself native or mutant. A parent polynucleotide or gene encodes for a parent protein or enzyme. In a certain embodiment, a “parent” protein, enzyme, polynucleotide, gene, or cell, is a wild type protein, enzyme, polynucleotide, gene, or cell.

“Isolated polypeptide” refers to a polypeptide which is separated from other contaminants that naturally accompany it, e.g., protein, lipids, and polynucleotides. The term embraces polypeptides which have been removed or purified from their naturally-occurring environment or expression system (e.g., host cell or in vitro synthesis).

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure polypeptide composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species.

“Reference sequence” refers to a defined sequence used as a basis for a sequence comparison. A reference sequence may be a subset of a larger sequence, for example, a segment of a full-length gene or polypeptide sequence. Generally, a reference sequence can be at least 20 nucleotide or amino acid residues in length, at least 25 nucleotide or residues in length, at least 50 nucleotides or residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptides are typically performed by comparing sequences of the two polynucleotides or polypeptides over a “comparison window” to identify and compare local regions of sequence similarity.

“Sequence identity” means that two amino acid sequences are substantially identical (i.e., on an amino acid-by-amino acid basis) over a window of comparison. The term “sequence similarity” refers to similar amino acids that share the same biophysical characteristics. The term “percentage of sequence identity” or “percentage of sequence similarity” is calculated by comparing two optimally aligned sequences over the window of comparison, determining the number of positions at which the identical residues (or similar residues) occur in both polypeptide sequences to yield the number of matched positions, dividing the number of matched positions by the total number of positions in the window of comparison (i.e., the window size), and multiplying the result by 100 to yield the percentage of sequence identity (or percentage of sequence similarity). With regard to polynucleotide sequences, the terms sequence identity and sequence similarity have comparable meaning as described for protein sequences, with the term “percentage of sequence identity” indicating that two polynucleotide sequences are identical (on a nucleotide-by-nucleotide basis) over a window of comparison. As such, a percentage of polynucleotide sequence identity (or percentage of polynucleotide sequence similarity, e.g., for silent mutations or other mutations, based upon the analysis algorithm) also can be calculated. Maximum correspondence can be determined by using one of the sequence algorithms described herein (or other algorithms available to those of ordinary skill in the art) or by visual inspection.

As applied to polypeptides, the term substantial identity or substantial similarity means that two peptide sequences, when optimally aligned, such as by the programs BLAST, GAP or BESTFIT using default gap weights or by visual inspection, share sequence identity or sequence similarity. Similarly, as applied in the context of two nucleic acids, the term substantial identity or substantial similarity means that the two nucleic acid sequences, when optimally aligned, such as by the programs BLAST, GAP or BESTFIT using default gap weights (described elsewhere herein) or by visual inspection, share sequence identity or sequence similarity.

One example of an algorithm that is suitable for determining percent sequence identity or sequence similarity is the FASTA algorithm, which is described in Pearson, W. R. & Lipman, D. J., (1988) Proc. Natl. Acad. Sci. USA 85:2444. See also, W. R. Pearson, (1996) Methods Enzymology 266:227-258. Preferred parameters used in a FASTA alignment of DNA sequences to calculate percent identity or percent similarity are optimized, BL50 Matrix 15: −5, k-tuple=2; joining penalty=40, optimization=28; gap penalty—12, gap length penalty=−2; and width=16.

Another example of a useful algorithm is PILEUP. PILEUP creates a multiple sequence alignment from a group of related sequences using progressive, pairwise alignments to show relationship and percent sequence identity or percent sequence similarity. It also plots a tree or dendogram showing the clustering relationships used to create the alignment. PILEUP uses a simplification of the progressive alignment method of Feng & Doolittle, (1987) J. Mol. Evol. 35:351-360. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity (or percent sequence similarity) relationship using the following parameters: default gap weight (3.00), default gap length weight (0.10), and weighted end gaps. PILEUP can be obtained from the GCG sequence analysis software package, e.g., version 7.0 (Devereaux et al., (1984) Nuc. Acids Res. 12:387-395).

Another example of an algorithm that is suitable for multiple DNA and amino acid sequence alignments is the CLUSTALW program (Thompson, J. D. et al., (1994) Nuc. Acids Res. 22:4673-4680). CLUSTALW performs multiple pairwise comparisons between groups of sequences and assembles them into a multiple alignment based on sequence identity. Gap open and Gap extension penalties were 10 and 0.05 respectively. For amino acid alignments, the BLOSUM algorithm can be used as a protein weight matrix (Henikoff and Henikoff, (1992) Proc. Natl. Acad. Sci. USA 89:10915-10919).

Additional favorable polypeptides sequences for engineering can be identified by using sequence alignment. For example, sequences that have sequence alignment of at least 80% to SEQ ID NO:1, SEQ ID NO:7 and/or the accession numbers listed above would provide for such sequences. The modified polypeptide may then be assayed for unnatural efficiency using the methods described herein.

Biomanufacturing, the synthesis of chemicals from renewable resources by engineered microbes, holds promise to transform the current fossil fuel-based chemical industry for a sustainable future. Although numerous fuels, pharmaceuticals, and commodities have been biomanufactured, the vast majority of these processes failed to proceed beyond lab scale because the productivity, titer, and yield are still low. This problem highlights the existence of a knowledge gap in the understanding of cell metabolism. This knowledge gap exists largely due to the extraordinary complexity of metabolic systems.

To overcome the complexity problem, one solution is to insulate the much simpler, engineered pathways in an orthogonal metabolic system which operates in parallel to the hosts' complex native metabolism. Catabolism and anabolism are the most universal orthogonal metabolic systems in nature. These two seemingly opposing processes coexist without interference largely because they each have a designated redox cofactor, NAD⁺ and NADP⁺, respectively. Therefore, it's been hypothesized that a third, orthogonal metabolic system can be established if one can introduce an unnatural redox cofactor inside the cells.

In addition to their applications in vivo, unnatural redox cofactors have also been explored as more cost-effective alternatives to NAD(P)⁺ during in vitro biotransformation, where purified enzymes are used to manufacture chemicals. The majority of industrial biotransformation processes developed to date involve installing specific chiral centers using oxidoreductase enzymes, which require redox cofactors. Analogs of NAD(P)⁺ with smaller sizes and simpler structures are more stable, easier to synthesize, and have faster mass transfer rate, which may greatly reduce the cost of in vitro biotransformation.

Enzymatic biotransformation has been regarded as a feasible solution to manufacture chiral chemicals in an affordable and environmentally friendly manner. In such processes, natural redox cofactors NAD(P)H are regenerated in situ by coupled enzymatic reactions. Recent studies have explored opportunities to replace the expensive and unstable natural redox cofactors with their simpler analogs. Compared to the natural redox cofactors, these noncanonical cofactors typically retain the catalytically-essential nicotinamide moiety, but they are smaller in size and easier to synthesize. In addition to lowering costs, they also offer important advantages including higher stability and a faster diffusion rate. However, despite numerous efforts, two major roadblocks still remain which have impeded the widespread utilization of noncanonical cofactors. First, most native enzymes have very low activities towards the simpler NAD(P)H analogs, which limits the scope of chemistry accessible. Second, an efficient and facile method to regenerate the reduced noncanonical cofactors has been elusive. Ideally, such a method should also be “plug-in ready” to the existing biotransformation processes. Moreover, shifting enzymes' cofactor preference toward unnatural redox cofactors remains a challenging task.

Previous efforts to discover noncanonical cofactor-utilizing enzymes with high throughput have largely employed colorimetric methods based on reactive dyes that signal the production of reduced cofactor. These approaches have involved 96-well plate-based assays with a relatively low throughput (10²-10⁴ per iteration), or required heat treatment of colonies on agar plate to inactivate background enzyme activity followed by in-situ color development which limits the target to thermophilic enzymes. Compared to colorimetric development, cell growth is a much more facile and higher-throughput (>10⁶ per iteration) readout. In addition, growth-based selection can be augmented with in vivo, continuous mutagenesis to more closely mimic the depth and scale of natural evolution that Nature explores in navigating protein sequence space to yield enzymes with natural-like levels of activity.

Nicotinamide mononucleotide (NMN⁺), a truncated version of the native nicotinamide cofactors, was found to be an efficient noncanonical redox cofactor. Compared with other simpler NAD⁺ mimetics, NMN⁺ can be produced renewably using low-cost feedstocks through biosynthetic pathways. More importantly, its polar structural features (particularly the phosphate) offer unique advantages for enzyme design. Based on the design principle of introducing novel polar contacts with the NMN⁺ phosphate, an engineered glucose dehydrogenase (GDH) was found to recycle NMN⁺ with extremely high total turnover number (TTN). Further, the GDH NMN⁺ noncanonical cofactor system was further found to maintain the high-flux central carbon metabolism in Escherichia coli to support cell growth.

Growth selection is a powerful tool in enzyme engineering due to its easy readout and unparallel throughput (>10⁶ per iteration), compared to 96-well plate-based or agar plate-based colorimetric methods (10²-10⁴ per iteration). Multiple growth-based selection platforms have been designed to engineer NAD(P)H dependent enzymes, where the unifying principle behind is that cells can only grow when the life-essential redox reactions have their cofactors recycled continuously in vivo. This principle has been expanded herein for the noncanonical redox cofactor NMN⁺. The life-essential redox reaction for production of reduced glutathione (GSH) (See FIG. IA) in E. coli was chosen. This redox reaction is required by E. coli in order to maintain the intracellular reducing environment and survive through oxidative stresses.

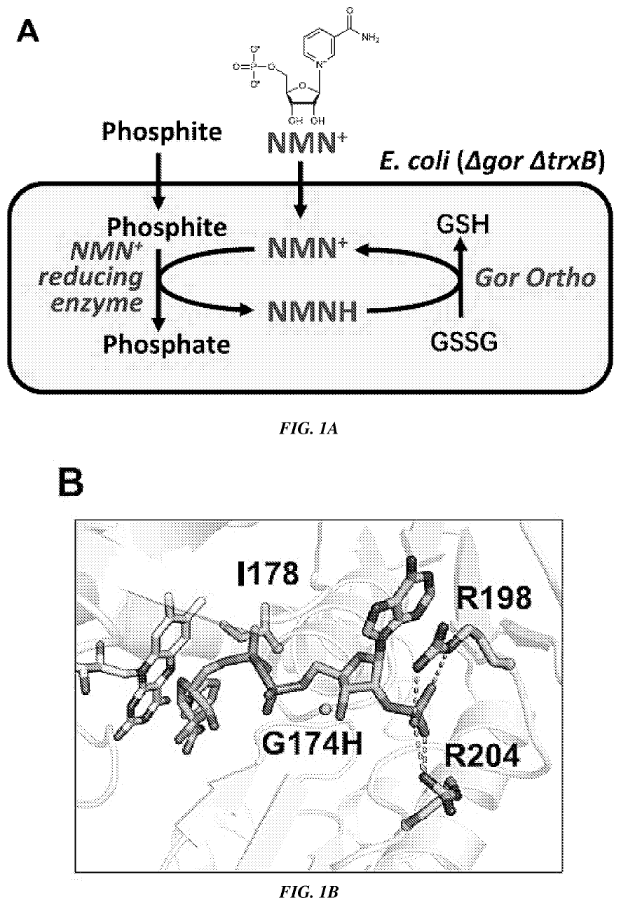

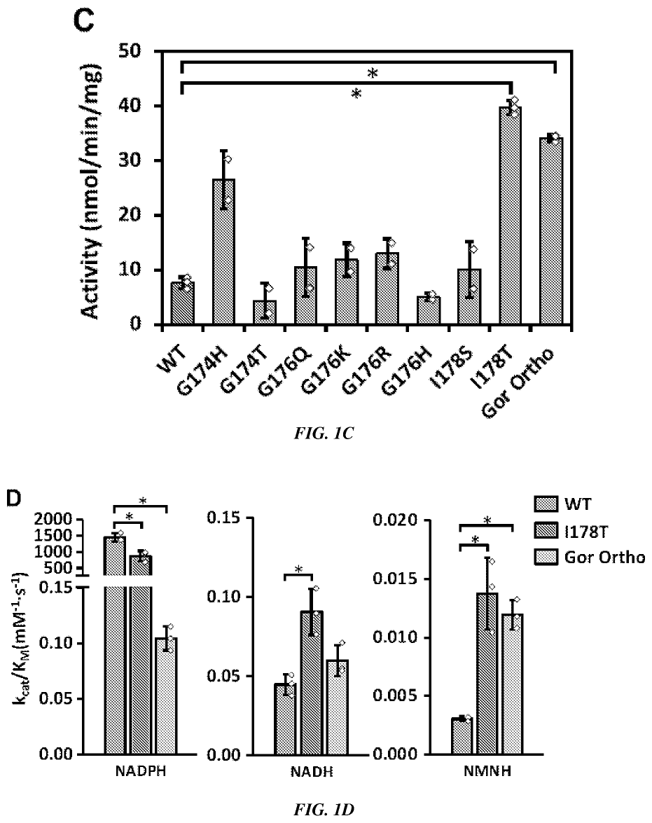

Reported herein it the development of a facile, high-throughput, and universal growth selection platform to obtain NMN⁺-utilizing enzymes through directed evolution. This platform will enable practical application of the noncanonical redox cofactor NMN on demand. Briefly, the growth selection is based on an engineered E. coli strain with a glutathione reductase (Gor) variant that makes the life-essential reduced glutathione (GSH) using reduced NMN⁺ (NMNH) as the reducing equivalent specifically (see FIG. 1A). When transformed with a library of enzyme variants, the selection strain will report the presence of any NMN⁺-reducing enzymes through growth as an easy readout. Using this selection platform, it was demonstrated herein the ability to evolve multiple efficient NMN⁺-recycling phosphite dehydrogenase (PTDH) variants, with up to ˜147-fold increased catalytic efficiency toward NMN⁺ compared to the wild type enzyme. In practice, these engineered PTDH variants offered a robust and cost-effective means of recycling NMN(H) in reduction biotransformation, achieving an industrially relevant TTN of ˜45,000 with NMN⁺. Furthermore, their low Kₘ values with NMN⁺ attained from growth selection enabled the PTDH variants to function near their maximum catalytic velocity, even at sub-millimolar cofactor concentrations, showcasing these PTDHs as transformative tools in reductive biotransformation. Interestingly, the engineered PTDHs also exhibit enhanced cycling of another even more cost-effective redox cofactor, 1-benzylnicotinamide (BNA⁺). Computational modeling of these variants revealed a common archetype in which the mutations occupy the binding pocket for the adenosine monophosphate (AMP) motif which is present in NAD(P)⁺ but missing in NMN⁺ and other simpler NAD⁺ biomimetics. It is envisaged that the selection platform disclosed herein can be used to evolve other noncanonical cofactor-dependent enzymes, because the redox balance-based mechanism is agnostic of the target enzyme's substrate and product.

There are multiple advantages of compositions, methods and kits of the disclosure. First, the glutathione reductase-based growth selection platform can be used to rapidly develop NMN⁺-utilizing enzymes, which may enable more economical and scalable biotransformation. Second, the PTDHs developed here are highly proficient catalysts in recycling noncanonical redox cofactors due to their superior TTN, ability to maintain turnover at low NMN⁺ concentrations, and capability to recycle other simpler cofactor biomimetics. Third, through deep searching of protein sequence space enabled by the high-throughput selection, a general design principle began to emerge which may shed light on the engineering of other noncanonical redox cofactor-dependent enzymes.

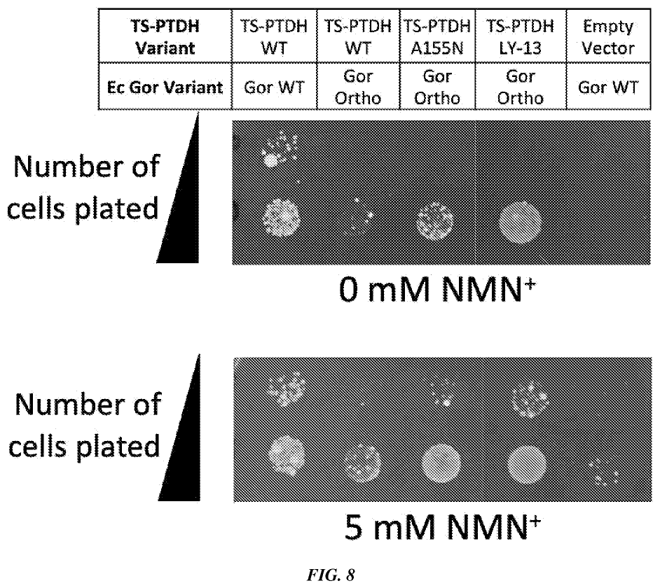

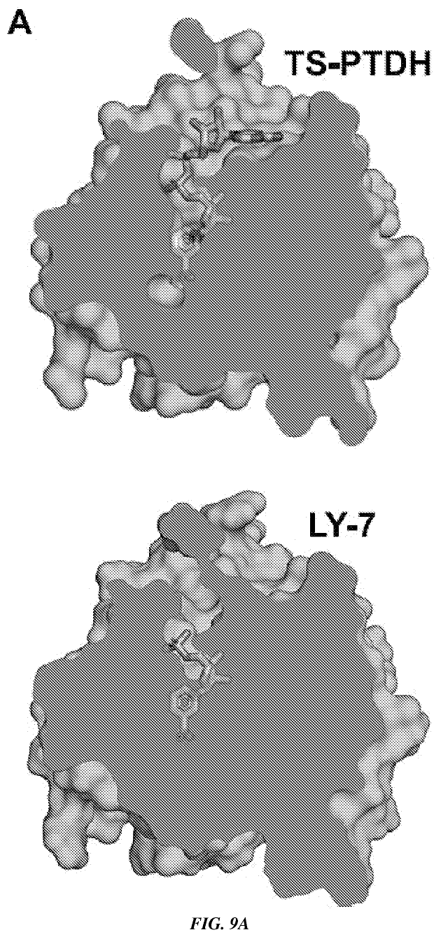

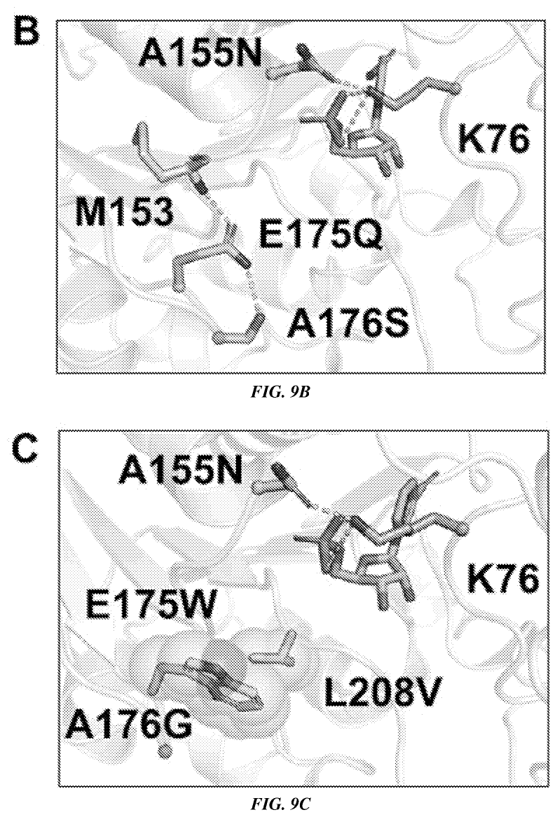

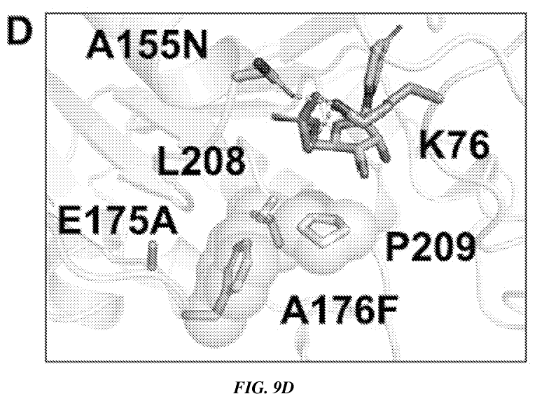

In a particular embodiment, the disclosure provides for the development of a NMNH-specific glutathione reductase. This NMNH-specific glutathione reductase was used to establish a high-throughput, growth-based selection platform to obtain NMN⁺-recycling PTDH variants through directed evolution. The selection platform was constructed by disrupting reduction of thiol-disulfides that interfere with protein folding in the cytoplasm of E. coli and directly eliminates low fitness variants unable to produce NMNH. Importantly, this growth selection platform enabled the directed evolution of enzymes for the reduction of NMN⁺. Compared to previously reported colorimetric assay-based approaches, utilizing growth as a simple readout afforded higher throughput which enabled the observation of a strong trend of convergent evolution: multiple NMN⁺-reducing variants employ very different sets of mutations to achieve the same predicted mechanism (recapitulating AMP) to enhance the NMN⁺-dependent activity. LY-6 utilized a hydrogen bond network to brace the AMP binding pocket, which is in stark contrast with the hydrophobic packing mode utilized by LY-7 and LY-13 (see FIG. 9). In addition, growth-based selection can be augmented with in vivo, continuous mutagenesis and adaptive laboratory evolution to more closely mimic the depth and scale of natural evolution that nature explores in navigating protein sequence space to yield enzymes with natural-like levels of activity. The high-throughput nature of the methods disclosed herein is particularly advantageous in shaping complex enzyme behavior, such as modulating conformational or allosteric dynamics.

Growth-based directed evolution is a powerful tool in enzyme design, but the main bottleneck is that cell metabolism must be tailored case-by-case to make the desired enzymatic activity essential for cell survival. This task is especially challenging when engineering enzymes for biotechnology applications, as most of the industrially important reactions neither exist in natural metabolism nor do they contribute to cell fitness. However, employing the redox balance principle can bypass this bottleneck in engineering redox cofactor-dependent enzyme. This disclosure provides that engineered glutathione reductase can be used as a universal reporter for intracellular availability of NMNH, to successfully distinguish high and low NMN⁺-reducing variants of two distinct enzymes, PTDH and GDH. The selection platform disclosed herein is readily adaptable to engineering other enzymes. The high-throughput nature of this tool is particularly advantageous in shaping complex enzyme behavior, such as modulating conformational or allosteric dynamics.

The AMP moiety of NAD(P)⁺ does not participate in redox reactions, but studies suggest that it may influence the enzymatic activity by modulating protein conformational dynamics. Indeed, crystallography studies on TS-PTDH reveal that the cofactor binding shifts the enzyme into a closed conformation, with a more compact active site and a better positioned K76 side chain for favorable interaction with the NMN⁺ phosphate. Therefore, it is possible that similar effects can be induced by the AMP-recapitulating mutations in the engineered PTDHs, which would be the basis for both the lowered KM toward NMN⁺ and the enhanced kcat. Structural studies and molecular dynamics simulations may be used to investigate this hypothesis. Interestingly, these AMP-recapitulating mutations also benefit BNA⁺ utilization. Therefore, this design principle may be added to the toolbox of rationally engineering BNA⁺-utilizing enzymes, in addition to the more obvious approach to directly contacting the benzyl recognition handle with strengthened van der Waals interactions.

Ultimately, the adoption of noncanonical redox cofactors in industrial processes is largely reliant on the availability of proteins capable of regenerating spent cofactors which are stable, utilize inexpensive substrates or waste streams, and catalytically active over a broad spectrum of reaction conditions. Ideally, these proteins exhibit low Kₘ and high k_{cat} values, enabling a highly productive system which operates at maximum efficiency over the course of their lifetime. However, fine tuning these specific parameters has largely proven difficult, especially when engineering for novel activity with noncanonical cofactors. The tunable, iterative, and inter-generational nature of this growth selection platform disclosed herein is uniquely suited to select for enzymes with improved catalytic efficiencies. In this disclosure, iterative rounds of growth selection with increasing selection pressure yielded a PTDH variant, LY-13, with robust temporal stability, TTN ˜45000, and a low Kₘ of 0.62 mM with NMN⁺. When applied to reductive biotransformation, the value of LY-13's low Kₘ was showcased by its ability to outperform GDH Triple, Kₘ=6.2 mM, at lower cofactor supplementation levels, despite GDH Triple's superior k_{cat}. Additional tuning of the growth selection pressure may enable even further improvements to these valuable biocatalysts. As reductive biotransformation processes scale, the synthetic cofactor BNA⁺ can be more cost-effective than NMN⁺. Curiously, the convergent adenine binding cleft mutations of LY-7 and LY-13 translate to enhanced activity for BNA⁺ reduction, despite a lack of specific interactions with its benzylic group. Further investigation through MD simulations may elucidate key design principles enabling biocatalysis across a broad scope of noncanonical redox cofactors.

Accordingly, the disclosure provides for recombinantly engineered polypeptides that have increased efficiency for an noncanonical cofactor than the wild-type protein or polypeptide, including a wild type protein that has a sequence of SEQ ID NO:1 or SEQ ID NO:7, or a thermostable variant of wild type protein that has a sequence of SEQ ID NO:2, or a wild type protein which has a sequence presented in an accession number of: WP_003246720.1, EHA28975.1, WP_119899028.1, CDH98271.1, WP_038427366.1, WP_095431766.1, WP_041340171.1, WP_032726518.1, AXV60254.1, WP_044161863.1, WP_014478842.1, WP_003225027.1, OTQ88242.1, WP_059291954.1, WP_010333037.1, KIU10883.1, WP_105991496.1, WP_095010766.1, ANW06331.1, PTU26434.1, WP_103749790.1, WP_077671287.1, WP_019713327.1, WP_014475815.1, AAA22463.1, WP_071581042.1, AGE62243.1, WP_103031562.1, WP_003240219.1, WP_071578344.1, WP_024714517.1, KJJ40202.1, WP_010330813.1, WP_064814593.1, WP_100741417.1, WP_087993024.1, WP_039075845.1, WP_070081367.1, WP_061522816.1, WP_098080985.1, WP_082998974.1, WP_088461430.1, WP_025284235.1, WP_061573960.1, WP_104678928.1, WP_061669578.1, WP_099744414.1, WP_065521908.1, WP_065980712.1, WP_106360802.1, WP_061184372.1, WP_073536545.1, WP_053403598.1, WP_000287801.1, WP_088119901.1, WP_000287802.1, WP_054768130.1, WP_061654990.1, WP_097824161.1, WP_098487332.1, WP_053485906.1, WP_000287797.1, WP_098607945.1, WP_043068355.1, WP_078417142.1, WP_048520053.1, WP_098671912.1, WP_098487331.1, WP_045294049.1, SUV21072.1, or WP_097856719.1.

For example, the disclosure provides for recombinantly engineered polypeptides that exhibit increased catalytic efficiency for noncanonical cofactors comprising a sequence that is: at least 85%, 90%, 95%, 98%, 99% identical to SEQ ID NO:1 or SEQ ID NO:2, wherein the sequence comprises a A155N substitution; at least 85%, 90%, 95%, 98%, 99% identical to SEQ ID NO:1 or SEQ ID NO:2, wherein the sequence comprises a E175Q, E175W, or E175Q mutation; at least 85%, 90%, 95%, 98%, 99% identical to SEQ ID NO:1 or SEQ ID NO:2, wherein the sequence comprises a A176S, A176G, or A176F substitution; at least 85%, 90%, 95%, 98%, 99% identical to SEQ ID NO:1 or SEQ ID NO:2, wherein the sequence comprises a L208V substitution; or at least 85%, 90%, 95%, 98%, 99% identical to SEQ ID NO:1 or SEQ ID NO:2, wherein the sequences comprises the following mutations: A155N, E175A, and A176F; wherein the foregoing polypeptides exhibit improved efficiency for noncanonical cofactors compared to their corresponding parental protein lacking said A155N, E175Q, El 75W, E175Q, A176S, A176G, A176F and/or L208V mutations.

The disclosure provides for recombinantly engineered polypeptides that exhibit increased catalytic efficiency for noncanonical cofactors comprising a sequence that is: at least 85%, 90%, 95%, 98%, 99% identical to SEQ ID NO:7, wherein the sequence comprises a I178T substitution; at least 85%, 90%, 95%, 98%, 99% identical to SEQ ID NO:7, wherein the sequence comprises a R198M substitution; at least 85%, 90%, 95%, 98%, 99% identical to SEQ ID NO:7, wherein the sequence comprises a R204L substitution; wherein the sequence comprises 2, or 3 of the following mutations: I178T, R198M, and R204L; wherein the foregoing polypeptides exhibit improved efficiency for noncanonical cofactors compared to their corresponding parental (wild-type) protein lacking said I178T, R198M, and R204L mutations.

In a further embodiment, the disclosure provides for recombinantly engineered polypeptides that exhibit increased efficiency for noncanonical cofactors comprising a sequence that is: at least 85%, 90%, 95%, 98%, 99% identical to SEQ ID NOs: 3, 4, 5, 6 or 8. In yet a further embodiment, the disclosure provides a direct evolution method as described herein for generating polypeptides that exhibit increased efficiency for noncanonical cofactors comprising a sequence that is: at least 85%, 90%, 95%, 98%, 99% identical to SEQ ID NOs: 1, 2, 3, 4, 5, 6 or 8, wherein mutations are generated based upon analysis of the sequences presented in SEQ ID NO: 3, 4, 5, 6 or 8 using Spartan and RosettaDesign. Additional favorable amino acid modifications can be engineered into the polypeptides based upon design considerations using Spartan and RosettaDesign. In yet a further embodiment, the disclosure provides using a growth selection process disclose herein for generating polypeptides that exhibit increased efficiency for noncanonical cofactors by using a recombinantly engineered polypeptide having the sequence of SEQ ID NO:8.

In a particular embodiment, an recombinantly engineered polypeptide of the disclosure exhibits a fold increase in catalytic efficiency towards the noncanonical cofactor in comparison to the corresponding parent or wild-type or parent polypeptide, or a thermostable variant thereof. In particular embodiment, the polypeptide of the disclosure has a fold increase of catalytic efficiency towards the noncanonical cofactor over the corresponding parent polypeptide of at least 100 fold, 200 fold, 300 fold, 400 fold, 500 fold, 600 fold, 700 fold, 800 fold, 900 fold, 1000 fold, 1500 fold, 2000 fold, 2500 fold, 3000 fold, 4000 fold, 5000 fold, 6000 fold, 7000 fold, 8000 fold, 9000 fold, 10000 fold, 15000 fold, 20000 fold, 50000 fold, 100000 fold, or a range that includes or is between any two of the foregoing values.

It has also been shown that the catalytic efficiencies for using the noncanonical cofactor can be predicted based on computer implemented protein design software. Such software, such as Spartan or RosettaDesign, allow for in silico modeling of coordinates and energies of the user designed proteins. For example, RosettaDesign searches for amino acid sequences that pack well, bury their hydrophobic atoms and satisfy the hydrogen bonding potential of polar atoms. RosettaDesign has been parameterized to return sequences with amino acid frequencies comparable to those found in naturally occurring proteins, and to partition the hydrophobic and polar residues between the surface and the core at naturally occurring frequencies. In general, when redesigning a naturally occurring protein ˜65% of the residues will mutate. As expected, more sequence variability is seen on the surface of the protein where there are fewer packing constraints. In the core of the protein 45% of the residues mutate on average. RosettaDesign can be used to help design new protein structures or portions of proteins. In this case, the user must supply the backbone coordinates of the target structure. However, an arbitrarily chosen protein backbones may not be designable.

Using the methods described herein a number of polypeptides have been engineered to have increased catalytic efficiency towards a noncanonical cofactor in comparison to the non-recombinantly engineered polypeptide or wild-type peptide.

In view of the general applicability of the design methods and techniques described herein, additional polypeptides could be generated using different parental sequences which exhibit increased oxidoreductase catalytic efficiency towards a noncanonical cofactor. Such different parental sequences could be selected from oxidoreductases and/or dehydrogenases which exhibit different substrate specificities; and oxidoreductases and dehydrogenases from different organisms or from chimeras generated thereof.

The disclosure further provides a growth selection process that can be used to identify recombinantly engineered polypeptide that exhibit increased activity for noncanonical cofactors. In a further embodiment, the growth selection process comprises the step of transforming microorganisms with: (i) a first recombinantly engineered polypeptide that can utilize a reduced form of a noncanonical cofactor for generating a reduced compound essential for bacterial survival and growth; and (ii) recombinantly engineered polypeptide(s) that comprise 1, 2, 3, 4, 5, 6, 7, 8, 9, 10, 11, or 12 introduced amino acid mutations in comparison to the sequence of the wild-type or parent polypeptide, or a thermostable variant thereof, wherein the introduced amino acid mutations are designed to increase the activity of the recombinantly engineered polypeptide(s) for an oxidized form of the noncanonical cofactor. In regards to the microorganisms, the microorganisms can be of any known family, including but not limited to, bacteria, archaea, and fungi (yeast). In a particular embodiment, the microorganism is a bacterium, such as E. coli. The microorganism may be first transformed with the first recombinantly engineered polypeptide and the recombinantly engineered polypeptide(s) sequentially or concurrently. In a particular embodiment, the first recombinantly engineered polypeptide comprises a sequence that is at least 98% or at least 99% identical to the sequence of SEQ ID NO: 8. The polypeptide sequence for the recombinantly engineered polypeptide(s) is typically comprises a sequence that is at least 85%, at least 90%, at least 95%, or at least 98% identical to a sequence from a dehydrogenase or an oxidoreductase. Examples of dehydrogenase or oxidoreductase sequences are provided above. The mutations introduced into the recombinantly engineered polypeptide(s) are typically designed based upon using a protein modelling program, such as RosettaDesign or Spartan.

The growth selection process further comprises a step of culturing the transformed microorganisms using a first selection criteria to identify variants with enhanced noncanonical-dependent activity, wherein the first selection criteria select for microorganisms that show enhanced noncanonical cofactor activity by exhibiting an increased growth or growth rate in comparison to other microorganisms; optionally, culturing the variants using additional selection criteria, wherein the additional selection criteria making it more, and more, challenging for the microorganisms to grow without having noncanonical cofactor activity. In yet another embodiment, the first selection criteria include providing culture conditions comprising the noncanonical cofactor, and selecting for variants that showed enhanced noncanonical factor-dependent activity. In a certain embodiment, the additional selection criteria include providing more challenging culture conditions that comprise the noncanonical cofactor, and a limited supply of a feedstock.

The disclosure further provides a cell-free system or whole-cell biomanufacturing systems to facilitate the biotransformation of a substrate into a desired product, comprising a recombinantly engineered polypeptide of the disclosure that has improved catalytic efficiency towards a noncanonical cofactor, and one or more polypeptides or proteins that encode enzymes that can use the same noncanonical cofactor in a biotransformation reaction(s). For example, if NMN⁺ (NHNH) is used as a noncanonical cofactor, then the cell-free system of the disclosure comprises an recombinantly engineered polypeptide disclosed herein that has improved catalytic efficiency towards NMN⁺ and a second recombinantly engineered polypeptide having the sequence of SEQ ID NO:8. Generally, the second recombinantly engineered polypeptide can utilize the reduced noncanonical cofactor being re-cycled by the recombinantly engineered polypeptides disclosed herein. It should be understood that the recombinantly engineered polypeptides of the disclosure are not just limited to NMN⁺ and in-fact can be engineered to have greater catalytic efficiencies for additional noncanonical cofactors, such as 1-phenyl-1,4,-dihydronicotinamide (PNA⁺), 1-benzyl-1,4-dihydronicotinamide (BNA⁺), 1-(4-hydroxyphenyl)1,4-dihydronicotinamide (HPNA⁺), 1-methyl-1,4-dihydronicotinamide (MNA⁺), nicotinamide flucytosine dinucleotide (NFCD⁺), nicotinamide mononucleoside (N12±), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile. For example, the studies presented herein show that some of the recombinantly engineered polypeptides of the disclosure have activity for BNA⁺.

The disclosure further provides a whole cell biomanufacturing system to facilitate the biotransformation of a substrate into a desired product, comprising recombinant microorganism (e.g., bacteria and yeast) that have been modified to express an recombinantly engineered polypeptide of the disclosure, i.e., a polypeptide that has improved catalytic efficiency towards an noncanonical cofactor and optionally express one or more polypeptides or proteins that encode enzymes that can use the same noncanonical cofactor in a biotransformation reaction(s). The recombinant microorganisms described herein, may further comprise one or more introduced mutations to affect the microorganisms' metabolic or enzymatic pathway(s), including, but not limiting to, introducing mutation(s) that disrupts one or more metabolic or enzymatic pathways of the microorganism, introducing one or more polypeptides that results in overexpression of one or more metabolic or enzymatic pathways of the microorganism, introducing one or more mutations that results in shunting metabolites from one metabolic or enzymatic pathway to another in the microorganism, introducing feedback mechanisms to either repress or activate enzymatic or metabolic pathways in the microorganism, or any combination of the foregoing. In the Examples presented herein, it was shown a biomanufacturing system comprising engineered E. coli cells that requires NMN⁺-based redox balance to grow. This growth phenotype enabled high-throughput selection, which allows for engineering NMN⁺-dependent enzymes through directed evolution, or optimizing NMN⁺-dependent pathways in vivo in a combinatorial manner. Similar redox balance-based, high-throughput selection platforms have been established for the two natural redox cofactors NAD⁺ and NADP⁺, which teachings of which indicate the possibilities of the biomanufacturing systems described herein (see e.g., Liang et al., Metabolic engineering 39, 181-191 (2017); Machado et al., Metabolic engineering 14, 504-511 (2012); and Zhang et al., ACS synthetic biology (2018).

The disclosure further provides that the methods and compositions described herein can be further defined by the following aspects (aspects 1 to 54):

1. A recombinantly engineered polypeptide that has improved catalytic efficiency for an oxidized form of a noncanonical cofactor, wherein the recombinantly engineered polypeptide comprises from 1 to 12 amino acid mutations in comparison to the sequence of the wild-type or parent polypeptide, or a thermostable variant thereof; wherein the amino acid mutations promote catalytic efficiency of the recombinantly engineered polypeptide in reducing the oxidized form of the noncanonical cofactor, and wherein a polypeptide having the sequence of the wild-type or parent polypeptide, or a thermostable variant thereof has minimal to very limited catalytic efficiency for the oxidized form of the noncanonical cofactor; wherein the recombinantly engineered polypeptide is configured to in vivo recycle the nonconical cofactor when used with a second recombinantly engineered polypeptide that utilizes the reduced form of noncanonical cofactor for cell growth, particularly wherein the recombinantly engineered polypeptide comprises 1, 2, 3, 4, 5, 6, 7, 8, 9, 10, 11 or 12 amino acid mutations in comparison to the sequence of the wild-type or parent polypeptide, or a thermostable variant thereof, particularly wherein the amino acid mutations are amino acid substitutions.

2. The recombinantly engineered polypeptide of aspect 1, wherein the recombinantly engineered polypeptide comprises from 3 to 8 amino acid mutations in comparison to the sequence of the wild-type or parent polypeptide, or a thermostable variant thereof, particularly wherein the recombinantly engineered polypeptide comprises 3, 4, 5, 6, 7, or 8 amino acid mutations in comparison to the sequence of the wild-type or parent polypeptide, or a thermostable variant thereof, more particularly wherein the recombinantly engineered polypeptide comprises from 3 to 6 amino acid mutations in comparison to the sequence of the wild-type or parent polypeptide, or a thermostable variant thereof, particularly wherein the amino acid mutations are amino acid substitutions.

3. The recombinantly engineered polypeptide of aspect 1 or aspect 2, wherein the amino acid mutations are amino acid substitutions.

4. The recombinantly engineered polypeptide of any one of the preceding aspects, wherein the recombinantly engineered polypeptide has at least 10-fold catalytic efficiency for the noncanonical cofactor in comparison to the wild-type or parent polypeptide, or a thermostable variant thereof.

5. The recombinantly engineered polypeptide of any one of the preceding aspects, wherein the recombinantly engineered polypeptide has at least 100-fold catalytic efficiency for the noncanonical cofactor in comparison to the wild-type or parent polypeptide, or a thermostable variant thereof.

6. The recombinantly engineered polypeptide of any one of the preceding aspects, wherein the recombinantly engineered polypeptide has at least 1000-fold catalytic efficiency for the noncanonical cofactor in comparison to the wild-type or parent polypeptide, or a thermostable variant thereof.

7. The recombinantly engineered polypeptide of any one of the preceding aspects, wherein the recombinantly engineered polypeptide further comprises from 1 to 8 additional amino acid mutations in comparison to the wild-type or parent polypeptide, or a thermostable variant thereof, wherein the additional amino acid mutations disrupt electrostatic complementarity between the recombinantly engineered polypeptide and a natural cofactor, particularly wherein the recombinantly engineered peptide further comprises 1, 2, 4, 5, 6, 7, or 8 additional amino acid mutations to the wild-type or parent polypeptide, or a thermostable variant thereof, particularly wherein the additional amino acid mutations are amino acid substitutions.

8. The recombinantly engineered polypeptide of aspect 7, wherein the additional amino acid mutations disrupt hydrogen bond formation between the recombinantly engineered polypeptide and a natural cofactor selected from flavin mononucleotide, flavin adenine dinucleotide, nicotinamide adenine dinucleotide, and nicotinamide adenine dinucleotide phosphate.

9. The recombinantly engineered polypeptide of aspect 7 or aspect 8, wherein the additional amino acid mutations disrupt hydrogen bond formation between the recombinantly engineered polypeptide and nicotinamide adenine dinucleotide.

10. The recombinantly engineered polypeptide of any one of the preceding aspects, wherein the noncanonical cofactor is selected from the group consisting of nicotinamide mononucleotide (NMN), 1-phenyl-1,4,-dihydronicotinamide (PNA), 1-benzyl-1,4-dihydronicotinamide (BNA), 1-(4-hydroxyphenyl)1,4-dihydronicotinamide (HPNA), 1-methyl-1,4-dihydronicotinamide (MNA), nicotinamide flucytosine dinucleotide (NFCD), nicotinamide mononucleoside (NR), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile.

11. The recombinantly engineered polypeptide of aspect 10, wherein the noncanonical cofactor is NMN or BNA.

12. The recombinantly engineered polypeptide of any one of the preceding aspects, wherein the recombinantly engineered polypeptide has a decrease of 30-fold or more in catalytic activity for the natural cofactor in comparison to the wild-type or parent polypeptide, or a thermostable variant thereof, particularly wherein the recombinantly engineered polypeptide has a decrease of 50-fold or more in catalytic activity for the natural cofactor in comparison to the wild-type or parent polypeptide, or a thermostable variant thereof, more particularly wherein the recombinantly engineered polypeptide has a decrease of 50-fold or more in catalytic activity for the natural cofactor in comparison to the wild-type or parent polypeptide, or a thermostable variant thereof.

13. The recombinantly engineered polypeptide of any one of the preceding aspects, wherein the recombinantly engineered polypeptide comprises a sequence that is at least 85% identical to a sequence from a dehydrogenase or an oxidoreductase, particularly wherein the recombinantly engineered polypeptide comprises a sequence that is at least 90% identical to a sequence from a dehydrogenase or an oxidoreductase.

14. The recombinantly engineered polypeptide of aspect 14, where the dehydrogenase or the oxidoreductase is selected from phosphite dehydrogenase (PTDH), alcohol dehydrogenase (NAD), alcohol dehydrogenase (NADP), glutathione reductase, homoserine dehydrogenase, glucose dehydrogenase, glycerol dehydrogenase, propanediol-phosphate dehydrogenase, glycerol-3-phosphate dehydrogenase (NAD⁺), lactate dehydrogenase, malate dehydrogenase, isocitrate dehydrogenase, acetaldehyde dehydrogenase, glyceraldehyde 3-phosphate dehydrogenase, pyruvate dehydrogenase, oxoglutarate dehydrogenase, phosphite and formate dehydrogenase.

15. The recombinantly engineered polypeptide of aspect 14, wherein the dehydrogenase is a PTDH.

16. The recombinantly engineered polypeptide of aspect 15, wherein the PTDH comprises sequence that is at least 98% identical to the sequence of SEQ ID NO:3, SEQ ID NO:4, SEQ ID NO:5, or SEQ ID NO:6.

17. A recombinantly engineered polypeptide that has improved catalytic efficiency for a reduced form of a noncanonical cofactor, wherein the recombinantly engineered polypeptide comprises from 3 to 12 amino acid mutations in comparison to the sequence of the wild-type or parent polypeptide, or a thermostable variant thereof; wherein the amino acid mutations promote catalytic efficiency of the recombinantly engineered polypeptide for the reduced form of the noncanonical cofactor and/or reduces catalytic activity for natural cofactor(s), and wherein a polypeptide having the sequence of the wild-type or parent polypeptide, or a thermostable variant thereof has minimal to very limited catalytic efficiency for the reduced form of the noncanonical cofactor; wherein the recombinantly engineered polypeptide uses the reduced noncanonical cofactor for cell growth, particularly wherein the recombinantly engineered polypeptide comprises 3, 4, 5, 6, 7, 8, 9, 10, 11 or 12 amino acid mutations in comparison to the sequence of the wild-type or parent polypeptide, or a thermostable variant thereof, particularly wherein the amino acid mutations are amino acid substitutions.

18. The recombinantly engineered polypeptide of aspect 17, wherein the recombinantly engineered polypeptide comprises from 3 to 8 amino acid mutations in comparison to the sequence of the wild-type or parent polypeptide, or a thermostable variant thereof, particularly wherein the recombinantly engineered polypeptide comprises 3, 4, 5, 6, 7, or 8 amino acid mutations in comparison to the sequence of the wild-type or parent polypeptide, or a thermostable variant thereof, more particularly wherein the recombinantly engineered polypeptide comprises from 3 to 6 amino acid mutations in comparison to the sequence of the wild-type or parent polypeptide, or a thermostable variant thereof, particularly wherein the amino acid mutations are amino acid substitutions.

19. The recombinantly engineered polypeptide of aspect 17 or aspect 18, wherein the amino acid mutations are amino acid substitutions.

20. The recombinantly engineered polypeptide of any one of aspects 17 to 19, wherein the recombinantly engineered polypeptide has at least 2-fold catalytic efficiency for the reduced form of the noncanonical cofactor in comparison to the wild-type or parent polypeptide, or a thermostable variant thereof.

21. The recombinantly engineered polypeptide of any one of aspects 17 to 20, wherein the recombinantly engineered polypeptide has at least 3-fold catalytic efficiency for the reduced form of the noncanonical cofactor in comparison to the wild-type or parent polypeptide, or a thermostable variant thereof.

22. The recombinantly engineered polypeptide of any one of aspects 17 to 21, wherein the recombinantly engineered polypeptide has at least 4-fold catalytic efficiency for the reduced form of the noncanonical cofactor in comparison to the wild-type or parent polypeptide, or a thermostable variant thereof.

23. The recombinantly engineered polypeptide of any one of aspects 17 to 22, wherein a portion of the amino acid mutations disrupt hydrogen bond formation between the recombinantly engineered polypeptide and a natural cofactor selected from flavin mononucleotide, flavin adenine dinucleotide, nicotinamide adenine dinucleotide, and nicotinamide adenine dinucleotide phosphate.

24. The recombinantly engineered polypeptide of any one of aspects 17 to 23, wherein the noncanonical cofactor is selected from the group consisting of nicotinamide mononucleotide (NMN), 1-phenyl-1,4,-dihydronicotinamide (PNA), 1-benzyl-1,4-dihydronicotinamide (BNA), 1-(4-hydroxyphenyl)1,4-dihydronicotinamide (HPNA), 1-methyl-1,4-dihydronicotinamide (MNA), nicotinamide flucytosine dinucleotide (NFCD), nicotinamide mononucleoside (NR), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile.

25. The recombinantly engineered polypeptide of aspect 24, wherein the noncanonical cofactor is NMN or BNA.

26. The recombinantly engineered polypeptide of any one of aspects 17 to 25, wherein the recombinantly engineered polypeptide has a decrease of 1,000-fold or more in catalytic activity for the natural cofactor in comparison to the wild-type or parent polypeptide, or a thermostable variant thereof.

27. The recombinantly engineered polypeptide of aspect 26, wherein the recombinantly engineered polypeptide has a decrease of 10,000-fold or more in catalytic activity for the natural cofactor in comparison to the wild-type or parent polypeptide, or a thermostable variant thereof.

28. The recombinantly engineered polypeptide of any one of aspects 17 to 27, wherein the recombinantly engineered polypeptide has at least a 20,000-fold cofactor specificity switch from a natural cofactor to a non-canonical cofactor, particularly wherein the recombinantly engineered polypeptide has at least a 40,000-fold cofactor specificity switch from a natural cofactor to a non-canonical cofactor, more particularly wherein the recombinantly engineered polypeptide has at least a 50,000-fold cofactor specificity switch from a natural cofactor to a non-canonical cofactor.

29. The recombinantly engineered polypeptide of any one of aspects 17 to 28, wherein the recombinantly engineered polypeptide comprises a sequence that is at least 85% identical to a sequence from a dehydrogenase or an oxidoreductase, particularly wherein the recombinantly engineered polypeptide comprises a sequence that is at least 90% identical to a sequence from a dehydrogenase or an oxidoreductase.

30. The recombinantly engineered polypeptide of aspect 29, wherein the oxidoreductase is a glutathione reductase.

31. The recombinantly engineered polypeptide of aspect 30, wherein the glutathione reductase comprises a sequence that is at least 98% identical to the sequence of SEQ ID NO:8, and has glutathione reductase activity.

32. The recombinantly engineered polypeptide of aspect 31 or aspect 32, wherein the glutathione reductase comprises the sequence of SEQ ID NO:8.

33. The recombinantly engineered polypeptide of any one of aspects 17 to 32, wherein the wild-type or parent polypeptide, or a thermostable variant thereof has the sequence of SEQ ID NO:7.

33. A growth selection process to identify recombinantly engineered polypeptides capable of using a noncanonical cofactor, the growth selection process comprising:
expressing in microorganisms:
   (i) a first recombinantly engineered polypeptide that comprises from 3 to 12 amino acid mutations in comparison to the sequence of the wild-type or parent polypeptide, or a thermostable variant thereof, wherein the amino acid mutations are designed to increase the activity of the recombinantly engineered polypeptide to utilize a reduced form of a noncanonical cofactor for microorganism growth; and
   (ii) a second recombinantly engineered polypeptide that comprises from 1 to 12 amino acid mutations in comparison to the sequence of the wild-type or parent polypeptide, or a thermostable variant thereof, wherein the amino acid mutations are designed to increase the activity of the recombinantly engineered polypeptide to reduce an oxidized form of the noncanonical cofactor;
culturing the transformed microorganisms using a first selection criteria to identify variants with enhanced noncanonical-dependent activity, wherein the first selection criteria select for variants that show enhanced noncanonical cofactor activity by exhibiting an increased growth or growth rate in comparison to other transformed microorganisms;
optionally, culturing the variants using additional selection criteria, wherein the additional selection criteria making it more, and more, challenging for the microorganisms to grow without having noncanonical cofactor activity.

34. The growth selection process of aspect 33, wherein the microorganisms are engineered to have an intracellular environment that is largely oxidative, by the disruption of gene(s) that encode protein(s) which reduce intracellular oxidative stress, particularly, wherein the gene for glutathione reductase (Gor) and/or the gene for thioredoxin reductase (TrxB) are disrupted.

35. The growth selection process of aspect 33 or aspect 34, wherein the first recombinantly engineered polypeptide comprises from 3 to 8 amino acid mutations in comparison to the sequence of the wild-type or parent polypeptide, or a thermostable variant thereof, particularly wherein the first recombinantly engineered polypeptide comprises 3, 4, 5, 6, 7, or 8 amino acid mutations in comparison to the sequence of the wild-type or parent polypeptide, or a thermostable variant thereof, more particularly wherein the first recombinantly engineered polypeptide comprises from 3 to 6 amino acid mutations in comparison to the sequence of the wild-type or parent polypeptide, or a thermostable variant thereof, particularly wherein the amino acid mutations are amino acid substitutions.

36. The growth selection process of any one of aspects 33 to 35, wherein the first recombinantly engineered polypeptide comprises from 3 to 8 amino acid mutations in comparison to the sequence of the wild-type or parent polypeptide, or a thermostable variant thereof, particularly wherein the recombinantly engineered polypeptide comprises 3, 4, 5, 6, 7, or 8 amino acid mutations in comparison to the sequence of the wild-type or parent polypeptide, or a thermostable variant thereof, more particularly wherein the recombinantly engineered polypeptide comprises from 3 to 6 amino acid mutations in comparison to the sequence of the wild-type or parent polypeptide, or a thermostable variant thereof, particularly wherein the amino acid mutations are amino acid substitutions.

37. The growth selection process of any one of aspects 33 to 36, wherein the first recombinantly engineered polypeptide has at least 2-fold catalytic efficiency for the reduced form of the noncanonical cofactor in comparison to the wild-type or parent polypeptide, or a thermostable variant thereof.

38. The growth selection process of any one of aspects 33 to 37, wherein the first recombinantly engineered polypeptide has at least 3-fold catalytic efficiency for the reduced form of the noncanonical cofactor in comparison to the wild-type or parent polypeptide, or a thermostable variant thereof.

39. The growth selection process of any one of aspects 33 to 38, wherein the recombinantly engineered polypeptide has at least 4-fold catalytic efficiency for the reduced form of the noncanonical cofactor in comparison to the wild-type or parent polypeptide, or a thermostable variant thereof.

40. The growth selection process of any one of aspects 33 to 39, wherein a portion of the amino acid mutations for the first recombinantly engineered polypeptide disrupt hydrogen bond formation between the recombinantly engineered polypeptide and a natural cofactor selected from flavin mononucleotide, flavin adenine dinucleotide, nicotinamide adenine dinucleotide, and nicotinamide adenine dinucleotide phosphate.

41. The growth selection process of any one of aspects 33 to 40,wherein the first recombinantly engineered polypeptide has a decrease of 1,000-fold or more in catalytic activity for the natural cofactor in comparison to the wild-type or parent polypeptide, or a thermostable variant thereof.

42. The growth selection process of aspect 41, wherein the recombinantly engineered polypeptide has a decrease of 10,000-fold or more in catalytic activity for the natural cofactor in comparison to the wild-type or parent polypeptide, or a thermostable variant thereof.

43. The growth selection process of any one of aspects 33 to 42, wherein the first recombinantly engineered polypeptide has at least a 20,000-fold cofactor specificity switch from a natural cofactor to a non-canonical cofactor, particularly wherein the first recombinantly engineered polypeptide has at least a 40,000-fold cofactor specificity switch from a natural cofactor to a non-canonical cofactor, more particularly wherein the first recombinantly engineered polypeptide has at least a 50,000-fold cofactor specificity switch from a natural cofactor to a non-canonical cofactor.

44. The growth selection process of any one of aspects 33 to 43, wherein the first recombinantly engineered polypeptide comprises a sequence that is at least 85% identical to a sequence from a dehydrogenase or an oxidoreductase, particularly, wherein the first recombinantly engineered polypeptide comprises a sequence that is at least 90% identical to a sequence from a dehydrogenase or an oxidoreductase.

45. The growth selection process of aspect 44, wherein the oxidoreductase is a glutathione reductase.

46. The growth selection process of aspect 45, wherein the glutathione reductase comprises a sequence that is at least 98% identical to the sequence of SEQ ID NO:8, and has glutathione reductase activity.

47. The growth selection process of aspect 45 or aspect 46, wherein the glutathione reductase comprises the sequence of SEQ ID NO:8.

48. The growth selection process of any one of aspects 33 to 47, wherein the amino acid mutations are selected by using a protein modeling program, particularly, wherein the protein modeling program predicts a protein structure de novo, more particularly, wherein the protein modeling program is selected from trRosetta, Robetta, Rosetta@home, Abalone, or C-Quark.

49. The growth selection process of any one of aspects 33 to 48, wherein the second recombinantly engineered polypeptide(s) comprises a sequence that is at least 85% identical to a sequence from a dehydrogenase or an oxidoreductase, particularly wherein the second recombinantly engineered polypeptide(s) comprises a sequence that is at least 90% identical to a sequence from a dehydrogenase or an oxidoreductase, more particularly, particularly wherein the second recombinantly engineered polypeptide(s) comprises a sequence that is at least 95% identical to a sequence from a dehydrogenase or an oxidoreductase.

50. The growth selection process of aspect 49, wherein the dehydrogenase or the oxidoreductase is selected from phosphite dehydrogenase (PTDH), alcohol dehydrogenase (NAD), alcohol dehydrogenase (NADP), glutathione reductase, homoserine dehydrogenase, glucose dehydrogenase, glycerol dehydrogenase, propanediol-phosphate dehydrogenase, glycerol-3-phosphate dehydrogenase (NAD⁺), lactate dehydrogenase, malate dehydrogenase, isocitrate dehydrogenase, acetaldehyde dehydrogenase, glyceraldehyde 3-phosphate dehydrogenase, pyruvate dehydrogenase, oxoglutarate dehydrogenase, phosphite and formate dehydrogenase.

51. The growth selection process of any one of aspects 33 to 50, wherein the noncanonical cofactor is selected from the group consisting of nicotinamide mononucleotide (NMN), 1-phenyl-1,4,-dihydronicotinamide (PNA), 1-benzyl-1,4-dihydronicotinamide (BNA), 1-(4-hydroxyphenyl)1,4-dihydronicotinamide (HPNA), 1-methyl-1,4-dihydronicotinamide (MNA), nicotinamide flucytosine dinucleotide (NFCD), nicotinamide mononucleoside (NR), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile.

52. The growth selection process of aspect 51, wherein the noncanonical cofactor is NMN or BNA.

53. The growth selection process of any one of aspects 33 to 52, wherein the first selection criteria include providing culture conditions comprising the noncanonical cofactor and an oxidizing agent, and selecting for variants that exhibit the greatest growth and/or growth rates, particularly, wherein the oxidizing agent is a thiol oxidizing agent, more particularly, wherein the oxidizing agent is selected from diamide or azoester.

54. The growth selection process of any one of aspects 33 to 53, wherein the second selection criteria include providing culture conditions of the first selection criteria but with a reduced concentration of the feedstock, and selecting for variants that exhibit the greatest growth and/or growth rates.

### EXAMPLES

Media and Growth Conditions. Bacterial strains and plasmids used in this disclosure are described in Table 1. The wild type Escherichia coli strain NEB express T7 and its derivative mutants SHuffle T7 Express (ΔtrxB, Δgor, ahpC*+cytoplasmic DsbC), were used for growth-based selection. XL-1 Blue was used to propagate all plasmids. BW25113 Δgor::kan obtained from the Yale E. coli Genetic Stock Center, was used to express Gor variants. BL21(DE3) was used to express all the other proteins. E. coli cells were cultured in 2×YT media containing 16 g/L tryptone, 10 g/L yeast extract, 5 g/L NaCl and appropriate antibiotics. M9 media contains 1 mM MgSO₄, 0.1 mM CaCl₂, trace metal mix A5 with Co (H₃BO₃ 2860 μg/L H₃BO₃ 2860 μg/L, MnCl₂·4H₂O 1810 μg/L, ZnSO₄ 7H₂O 222 μg/L, Na₂MoO₄, 2H₂O 390 μg/L, CuSO₄, 5H₂O 79 μg/L, Co(NO₃)₂.6H₂O (49 μg/L), and BD Difco M9 salts (Na₂HPO₄ 6.78 g/L, KH₂PO₄ 3 g/L, NaCl 0.5 g/L, NH₄Cl 1 g/L). Concentrations utilized for antibiotic selection were 100 mg/L for ampicillin, 50 mg/L for spectinomycin, 50 mg/L for kanamycin, and 10 mg/L for chloramphenicol. Induction was initiated with final concentrations of 0.5% arabinose for strains with P_{BAD} promoter, and 0.5 mM Isopropyl-β-D-thiogalactopyranoside (IPTG) for strains with P_{LlacO1} promoter. All strains were cultured at 37° C. with 250 r.p.m. agitation unless otherwise noted. Cell growth and enzyme assay were collected using a SpectraMax plate reader with SoftMax Pro 7.0 software.

TABLE 1
Stains and plasmids used in this study

| | Description |
|---|---|
| Strains | |
| XL-1 blue | Cloning strain |
| BL21 | Protein expression strain |
| (DE3) | |
| SHuffle T7 | E. coli BL21 fhuA2 lacZ::T7 gene1 [lon] ompT ahpC gal |
| express | λatt::pNEB3-r1-cDsbC (Spec^{R}, lacI^{q}) ΔtrxB sulA11 R(mcr- |
| | 73::miniTn10-Tet^{S})2 [dcm] R(zgb-210::Tn10-Tet^{S}) endA1 |
| | Δgor Δ(mcrC-mrr)114::IS10 |
| DH10β | Electrotransformation strain |
| JW3467-1 | BW25113 ΔGor::kan |
| Plasmids | |
| pQElac | P_{LlacO1}, ColE1 ori, Amp^{R}. Expression vector |
| pQElac gap | P_{LlacO1 gap}, ColE1 ori, Amp^{R}. Expression vector |
| pEK101 | P_{LlacO1}::Bs gdh, ColE1 ori, Amp^{R} |
| pLZ210 | P_{LlacO1}::Bs gdh I195R-A93K-Y39Q, ColE1 ori, Amp^{R} |
| pSM109 | P_{LlacO1}::Zm glf, p15A ori, Cm^{R} |
| pLZ301 | P_{LlacO1}::Ec gor, ColE1 ori, Amp^{R} |
| pLZ302 | P_{LlacO1}::Ec gor I174H, ColE1 ori, Amp^{R} |
| pLZ303 | P_{LlacO1}::Ec gor I174T, ColE1 ori, Amp^{R} |
| pLZ304 | P_{LlacO1}::Ec gor I176Q, ColE1 ori, Amp^{R} |
| pLZ305 | P_{LlacO1}::Ec gor I176K, ColE1 ori, Amp^{R} |
| pLZ306 | P_{LlacO1}::Ec gor I176R, ColE1 ori, Amp^{R} |
| pLZ307 | P_{LlacO1}::Ec gor I176H, ColE1 ori, Amp^{R} |
| pLZ308 | P_{LlacO1}::Ec gor I178S, ColE1 ori, Amp^{R} |
| pLZ309 | P_{LlacO1}::Ec gor I178T, ColE1 ori, Amp^{R} |
| pLZ310 | P_{LlacO1}::Ec gor I178T-R198M-R204L, ColE1 ori, Amp^{R} |
| pLZ311 | P_{BAD}:: Ec gor, RSF ori, Kan^{R} |
| pLZ312 | P_{BAD}:: Ec gor I178T-R198M-R204L, RSF ori, Kan^{R} |
| pEK201 | P_{LlacO1}:: Pst TS PTDH, ColE1 ori, Amp^{R} |
| pEK202 | P_{LlacO1}:: Pst TS PTDH A155N, ColE1 ori, Amp^{R} |
| pEK203 | P_{LlacO1}::Pst TS PTDH A155N-E175Q, ColE1 ori, Amp^{R} |
| pLZ313 | pQElac gap Pst TS PTDH, ColE1 ori, Amp^{R} |
| pLZ314 | pQElac gap Pst TS PTDH A155N, ColE1 ori, Amp^{R} |
| pLZ315 | pQElac gap Pst TS PTDH A155N-E175Q, ColE1 ori, |
| | Amp^{R} |
| pLZ316 | pQElac gap Pst TS PTDH A155N-E175-A176-L208 NNK |
| | library, ColE1 ori, Amp^{R} |
| pLZ317 | pQElac gap Pst TS PTDH A155N-E175Q-A176S, ColE1 |
| | ori, Amp^{R} |
| pLZ318 | pQElac gap Pst TS PTDH A155N-E175W-A176G-L208V, |
| | ColE1 ori, Amp^{R} |
| pLZ319 | pQElac gap Pst TS PTDH A155N-E175A-A176F, ColE1 |
| | ori, Amp^{R} |
| pLZ320 | P_{LlacO1}::Pst TS PTDH A155N-E175Q-A176S, ColE1 ori, |
| | Amp^{R} |
| pLZ321 | P_{LlacO1}::Pst TS PTDH A155N-E175W-A176G-L208V, |
| | ColE1 ori, Amp^{R} |
| pLZ322 | P_{LlacO1}::Pst TS PTDH A155N-E175A-A176F, ColE1 ori, |
| | Amp^{R} |

Abbreviations indicate source of genes: Bs, Bacillus subtilis; Zm, Zymomonas mobilis; Ec, Escherichia coli; Pst, Pseudomonas stutzeri.

Plasmid and TS-PTDH library Construction. The E. coli glutathione reductase (Gor) gene was amplified from E. coli BW25113 chromosomal DNA. The TS-PTDH was amplified from a synthesized DNA template (Integrated DNA Technologies, San Diego, CA). PCR fragments were generated using PrimeSTAR Max DNA Polymerase (TaKaRa) unless otherwise noted. After PCR and gel extraction, gene fragments were assembled with vector backbones (pQElac gap Amp^{R}, pRSF ori, Kan^{R}, or pQE Amp^{R}) using Gibson isothermal DNA assembly method, resulting in plasmids pLZ 301, 311, 313 and pEK 201. Plasmids carrying the Gor or PTDH mutations were generated using the corresponding wild type plasmids as a template and using site-directed mutagenesis to introduce single or multiple mutations.

The PTDH combinatorial site-saturation mutagenesis library pLZ316 was constructed with degenerate codon-containing primers. Briefly, PCR was used to amplify a DNA fragment from pLZ314 using a forward primer containing degenerate codons NNK at E175 and A176 positions together with a reverse primer containing degenerate codons MNN at L208. These two fragments were used as templates in a splicing-by-overlap extension PCR. The resulting fragment was assembled with a complementary fragment that was generated by PCR of the same template to amplify the rest of pLZ314 by Gibson assembly. The assembled plasmid was transformed into ElectroMAX DH10B cells (Invitrogen) by electroporation. Subsequently, the cells were rescued with SOC media at 37° C. for 1 hour with shaking and added into 20 mL 2×YT media with ampicillin. 0.2, 1 and 5 μL of culture was immediately taken from the culture and plated on an 2×YT agar plates with ampicillin. The plate was incubated at 37° C. overnight, and the number of colonies formed was counted to estimate the library size. The liquid culture was incubated at 37° C. with shaking for 10 h, the library DNA was extracted using QlAprep Spin Miniprep Kit (Qiagen). Six single colonies from the library estimation plates were cultured individually to extract plasmids, which were sequenced as representatives of the population. The results showed that all six plasmids contained unique mutation patterns, and no other mutations outside the intended mutagenesis sites were observed. The library size of pLZ316 was counted as 4.7×10⁷ transformants.

Expression and Purification of Gor Wild-type and Variants. E. coli Gor (EC 1.6.4.2) is a homo-dimeric flavoenzyme containing 450 amino acid residues with 1 FAD per subunit. To prevent interference from endogenous protein, Gor wild-type and variants were expressed using E. coli Δgor::kan strain containing pQElac based plasmids encoding for the Gor wild-type or variants. 1% (v/v) cells from an overnight culture were cultured in 2×YT media with 200 μg/mL ampicillin and 50 μg/mL kanamycin in shake flasks at 37° C. in a rotary shaker to an OD₆₀₀ of 0.4-0.6. The cultures were then induced with 0.5 mM IPTG and incubated at 30° C. with shaking for 24 h. The cells were harvested with centrifugation and lysed by bead beating with Zymo His Binding Buffer (Zymo Research, CA, USA). Protein purification was performed with Zymo His-Spin Protein miniprep purification kit according to the manufacturer's instructions. The concentrations of purified protein were quantified by Coomassie dye—based assays (Bradford) using BSA (Bovine Serum Albumin) as standards.

Specific Activity and Steady State Kinetic Analyses for Gor Wild-type and Variants. The specific activity and kinetic parameters of Gor with the different cofactors, shown in FIG. 1, were measured using the following methods. For measuring the specific activity with NMNH, the 100 μL reaction mixture contained 96 mM potassium phosphate buffer pH 7.5, 2.5 mM GSSG and 0.8 mM NMNH. NMNH was generated using methods described in Black et al. (Nat. Chem. Biol. 16:87-94 (2020). Briefly, 4 mM NMN⁺ was incubated in a solution containing 50 mM Tris-Cl at pH 8.5, 0.2 mM EDTA, 50 mM Na₂HPO₄, 5 mM D-glyceraldehyde-3-phosphate, and 28 μM of purified E. coli glyceraldehyde-3-phosphate dehydrogenase. The reaction was incubated at 30° C. for 8 h. The protein was removed by an Amicon Ultra 3 K filter, and the flow-through was dried under vacuum. Assays investigating the steady-state kinetic parameters were completed with 100 μL reaction mixtures containing 96 mM Tris-Cl buffer pH 7.5, 2.5 mM GSSG and varied cofactor concentrations from 0.01 mM to 1.6 mM. The assay was started by addition of 10 μL of purified protein at appropriate concentration at 25° C. The kinetic parameters were measured by monitoring the NAD(P)H consumption at 340 nm in 96-well plate. Data was fit to the Michaelis-Menten equation to generate estimates of apparent catalytic efficiency (k_{cat}/Kₘ). The results are the average of three independent experiments.

Culture conditions for GDH-Gor coupled growth rescue in SHuffle strain. The electro-competent cells of SHuffle harboring pLZ311 or pLZ312 were made as follows: The SHuffle cells were cultured in 200 mL SOB media with spectinomycin at 30° C. with shaking until OD₆₀₀ reached 0.4-0.6. The culture was chilled on ice for 15 min and the cells were pelleted at 4° C., 4,000×g. Collected cells were washed with 10% glycerol in water (sterile, ice cold) and resuspended with 5 mL of 10% glycerol in water (sterile, ice cold), and stored as 50 μL aliquots at −80° C.

Glucose facilitator (encoded by glf) was used for uptake of unphosphorylated glucose into the cells. The co-transformation of Bs gdh and Zm glf was performed as follows: Thaw SHuffle/pLZ311 or SHuffle/pLZ312 electro-competent cells on ice, add 1 μL Bs gdh (pEK101 or pLZ210) and 1 μL Zm glf(pSM109) plasmid DNA to 50 μL competent cells, mix, and electroporate. Cells were recovered with 1 mL SOC at 37° C. with shaking for 1 h. The cells were added to 10 mL 2×YT with 10 g/L glucose, 0.5% arabinose, kanamycin, chloramphenicol, and ampicillin. After incubation at 37° C. with shaking for 10 h, 1 mL cells were washed 3 times and re-suspended in M9 buffer (1×BD Difco M9 Salts). Targeted dilutions after cells reached density of OD₆₀₀ 0.6 were prepared in M9 buffer and 5 μL aliquots were dispensed in series on an agar plate of M9 selection media containing 0.5 mM diamide, 1 g/L yeast extract, 10 g/L glucose, 0.5% arabinose, 0.5 mM IPTG, ampicillin, kanamycin, chloramphenicol and serial NMN concentrations of 0, 2, 4 and 6 mM. Selection plates were incubated at 37° C., and photos were taken to document growth progress.

Selection of TS-PTDH Library. A total of 50 μL pre-made SHuffle/pLZ311 or SHuffle/pLZ312 electro-competent cells were transformed with 2 μL TS-PTDH A155N-E175-A176-L208 NNK library DNA (pLZ316) via electroporation as described above. After rescue in SOC media for 1 h, cultures were combined in 10 mL 2×YT with 10 g/L glucose, 0.5% arabinose, kanamycin and ampicillin. 0.2, or 1 μL of culture was immediately taken from the culture and plated on an 2×YT agar plates with kanamycin and ampicillin. The plate was incubated at 37° C. overnight, and the number of colonies formed was counted to estimate the library size. The liquid cultures were incubated at 37° C. with shaking for 10 h. Cells (1 mL) were washed 3 times and re-suspended in M9 Buffer. Targeted serial dilutions of cells were prepared in M9 buffer and 5 μL aliquots were dispensed in series on an agar plate of M9 selection media containing 0.5 mM diamide, 5 or 1 g/L yeast extract, 10 g/L glucose, 0.5% arabinose, 0.5 mM IPTG, 10 g/L sodium phosphite, kanamycin, ampicillin, and 5 mM NMN⁺. Selection plates were incubated at 37° C., and photographs were taken to document growth progress. The independent transformants sampled was calculated as 2.4×10⁶ and 2.5×10⁶ colonies when plated on 5 and 1 g/L yeast extract, respectively. TS-PTDH and the template of the library, TS-PTDH A155N, were cultured in the same conditions as controls. The colonies were numbered by the order of appearance. The selected cells were re-streaked on fresh agar plate with identical media and incubated until single colonies formed on the plate. Variants that cannot forma single colony may be false positives. After extracting the plasmids from each re-streaked colony, the chosen TS-PTDH variants were amplified and moved into the pQElac vector containing a N-terminal 6×His tag for protein purification and characterization. To demonstrate recapitulation of the growth phenotype, purified TS-PTDH and Gor variant plasmids were transformed identically to the growth selection system. 2 μL of a serial dilution of cells were deposited onto a growth selection plate and grown at 37° C.

Purification and Characterization of TS-PTDH Variants. The E. coli BL21(DE3) strains harboring pQElac plasmids encoding the TS-PTDH or TS-PTDH variants were grown in 2×YT media with kanamycin and ampicillin at 37° C. The IPTG-inducible expression, Ni-affinity purification, and protein concentration determination was conducted as described above.

The specific activities of TS-PTDH and variants were measured with the following methods. Briefly, the reaction mixture contained 100 mM MOPS buffer, pH 7.25, 10 mM sodium Pt, and 4 mM cofactor (NAD⁺, NADP⁺, or NMN⁺). The formation of reduced coenzyme was measured by light absorption at 340 nm in a 96-well plate at 25° C. Absorption was correlated to a molar basis using the extinction coefficient (ε) of 6.22 mM⁻¹ cm⁻¹ for each cofactor.

The steady-state kinetic parameters toward the coenzymes were measured by spiking purified protein into a 100 μL reaction mixture containing 100 mM MOPS, pH 7.25, 10 mM sodium Pt, and varied cofactor concentrations from 0.02 mM to 5 mM at 25° C. The kinetic parameters were measured by monitoring the consumption of reduced coenzyme at 340 nm. Data were fitted to the Michaelis-Menten equation to generate estimates of K_{M} and k_{cat} values.

Molecular modeling. Homology models for mutant TS-PTDH and Ec Gor were generated with Rosetta. Protein structures were visualized with Schrodinger PyMOL software. The crystal structure used as template for TS-PTDH had PDB identifier 4E5N, and the template for EC Gor was 1GET. Coordinates for NMN/H used in ligand docking were extracted from the co-crystallized nicotinamide cofactor in each template, NAD⁺ for 4E5N and NADP⁺ for 1GET, and the remaining AMP atoms were deleted. The Rosetta docking protocol involved mutation from the WT structure, repeated rounds of random rigid-body perturbation by translation and rotation for NMN/H, and optimization of active site rotamers through sidechain repacking and minimization. All moves were sampled with the Monte Carlo method, coordinate restraints were utilized to maintain the NMN/H in a catalytically competent pose, and full flexibility for ligand and protein backbone torsions was allowed to identify the optimal binding pose. A total of 1,000 docking trials was run for each variant, the top 100 models based on total Rosetta energy (an aggregate of residue energy terms describing van der Waals, electrostatics, rotamer conformation probabilities, etc. that serves as an indicator of complex stability) were sorted on interface energy scores (the difference in energy with the ligand separated from the complex that reflects the predicted favorability of ligand binding) and the model with the most favorable interface energy to NMN/H was selected as the reference following visual inspection to ensure no structural artifacts.

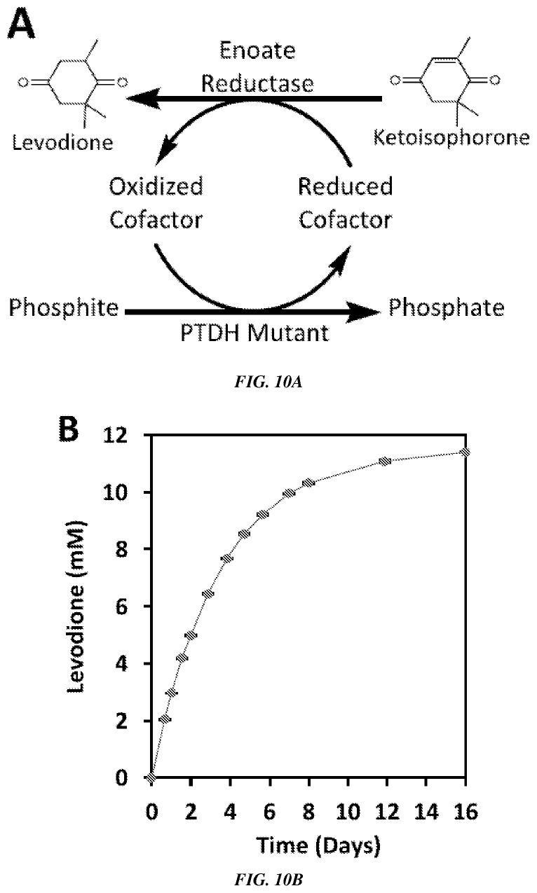

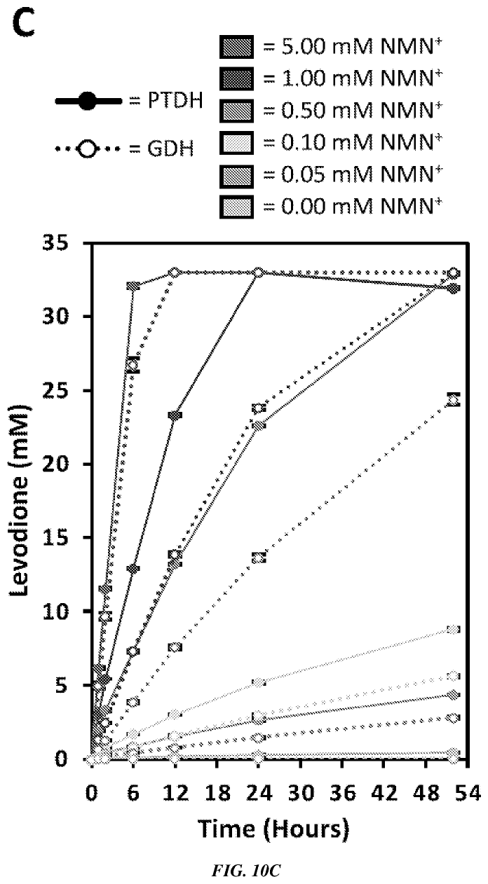

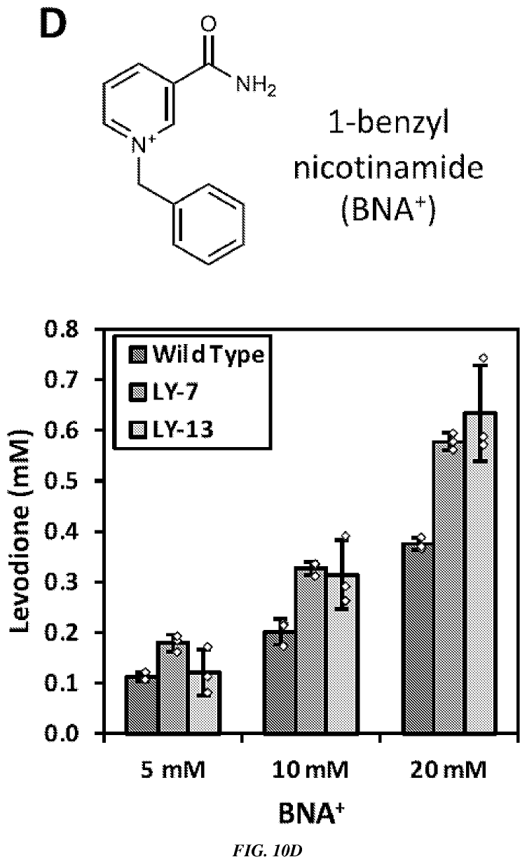

TS-PTDH Total Turnover Number Determination. Reactions to determine the TTN of TS-PTDH LY-13 (see FIG. 10B) were performed at 1 mL volumes in 2 mL glass vials secured with a PTFE-lined screw cap. Reactions were incubated without shaking at 30° C. Reaction buffer was comprised of 200 mM MOPS at pH 7.5, 100 mM sodium phosphite, 6 mM NMN⁺, and 33 mM ketoisophorone (KIP). To initiate the reaction, purified TS-PTDH LY-13 and XenA were spiked into the reaction mixture to a final concentration of 0.25 μM and 18.8 μM, respectively. The reactions were sampled intermittently by removing 50 μL of reaction mixture and mixing with 100 μL of ethyl acetate containing 200 mg/L1-octanol. Samples were vortexed, centrifuged, and the ethyl acetate fraction was transferred to a GC vial for GC-FID analysis. GC-FID analysis for KIP biotransformation efficiency was performed on an Agilent 6850 GD using an Agilent DB-WAX column (30 m×0.56 mm×1 μm). Helium was used as a carrier gas. The inlet and detector were held at 250 and 260° C., respectively. 5 μL of sample was injected with a 2:1 split ratio. The oven was held at 200° C. for 15 min. TS-PTDH LY-13 was added at a low concentration to require many turnovers of LY-13 for the reaction to progress. TTN was calculated by the moles of levodione produced divided by the moles of LY-13 supplied to the reaction system. Total turnover frequency was determined by dividing TTN by the total reaction time of 384 h. Levodione concentration was determined by correlating sample response to a standard curve of known values.

Comparison of PTDH and GDH Triple Redox Cofactor Cycling Systems. In cycling reactions focused on higher conversion and productivity, see FIG. 10C, the concentration of cofactor cycling protein (TS-PTDH or GDH Triple) was increased relative to the TTN determination experiments. Reactions were performed at 30° C. without shaking in 1 mL volumes in 2 mL glass vials secured with a PTFE-lined screw cap. Sampling and GC-FID analysis were performed as described in the TTN method section.

Reactions utilizing TS-PTDH LY-13 contained 100 mM MOPS at pH 7.5, 100 mM sodium phosphite, 33 mM KIP, and 18.8 μM XenA. Reactions utilizing GDH Triple contained 200 mM potassium phosphate at pH 7.5, 300 mM D-glucose, 1 M NaCl, 33 mM KIP, and 18.8 μM XenA. The NMN⁺ concentration in each reaction was varied at 0, 0.05, 0.1, 0.5, or 5 mM. The reactions were initiated by spiking TS-PTDH LY-13 or GDH Triple to a final concentration of 10 μM.

BNA(H)-Mediated PTDH Biotransformation. Reactions with BNA⁺, see FIG. 10D, contained 100 mM MOPS-NaOH at pH 7.6, 10 mM KIP, 100 mM sodium phosphite, and 2% v/v DMSO. BNA⁺Cl⁻, prepared by refluxing equimolar amounts of nicotinamide and benzyl chloride in acetonitrile overnight, followed by precipitation with diethyl ether on ice was supplied to the reactions at final concentrations of 0, 5, 10, or 20 mM. Reactions were initiated by spiking TsOYE and the appropriate TS-PTDH variant to a final concentration of 10 μM, each. Reaction mixtures were incubated at 30° C. on a Thermomixer C (Eppendorf) with shaking at 900 rpm for 24 h, extracted with 500 μL ethyl acetate containing 5 mM n-dodecane, and analyzed by GC-FID. The cycling system with no BNA was used to determine the background activity of the cycling systems. This value was subtracted from the final levodione concentrations in the 5-, 10-, and 20-mM samples to determine the BNA(H)⁻ specific levodione production. Concentrations of levodione product were determined using a calibration line (0-10 mM, 6 levels, using n-dodecane as internal standard). The y-intercept of the calibration line was forced through 0.

For BNA(H)-mediated reactions, gas chromatography analyses were performed on a GC-2010 (Shimadzu, Japan) equipped with an AOC-20i auto injector and a flame ionization detector (FID), using a CP-Sil 5 column (25 m×0.25 mm×1.2 μM). 1 μL of sample was injected with a split ratio of 100:1 and injector at 340° C. The FID was maintained at 360° C. Nitrogen was used as the carrier gas, with a linear velocity of 30 cm/s. The oven was held at 135° C. for 1 min, ramped at 15° C./min to 215° C., then 30° C./min to 345° C., and held at 345° C. for 1 min.

Expression and Purification of Proteins for BNA(H)-Mediated Biotransformation Reactions. E. coli BL21(DE3) strains were transformed with the plasmids containing the TS-PTDH variants and grown on LB-agar plates containing ampicillin at 37° C. overnight. An overnight culture, 15 mL LB supplemented with ampicillin, was inoculated with a single colony, and grown at 37° C., 170 rpm overnight. 2 L baffled shake-flasks with 250 mL of 2×YT media containing 200 μg/mL ampicillin, were inoculated with the overnight culture, grown at 37° C., 170 rpm until OD₆₀₀ of ˜1.0, induced with IPTG (0.5 mM), and grown at 30° C. and 170 rpm for 24 h. Cells were harvested by centrifugation at 18,000×g, 4° C., 10 min and stored at −20° C. until purification.

The pellet was resuspended in four volumes loading buffer (50 mM sodium phosphate, 300 mM sodium chloride, 10 mM imidazole, pH 7.7), and lysed on ice by sonication (Branson sonifier 250; 15 min total, 40% duty cycle, output control 3.5, 2 mm tip). The lysate was clarified (21,000×g, 4° C., 30 min followed by 0.2 μm filtration), and loaded (1 mL/min) onto a Nickel-HisTrap FF crude (1 mL) column, washed with loading buffer (12 column volumes, CV), followed by a step with 10% elution buffer (loading buffer containing 250 mM imidazole; 12 CV). The protein was then eluted with 100% elution buffer and collected in fractions of 0.5 mL. Fractions containing protein were combined and dialyzed against ice-cold dialysis buffer (50 mM sodium phosphate, pH 7.7, 800 mL); the buffer was renewed after the first 2 h of dialysis. Following the dialysis, the protein was mixed with glycerol (20% v/v final concentration). Enzyme concentrations were estimated by the absorbance at 280 nm (non-denatured protein), using predicted extinction coefficients (wild type and LY-13: 26470, LY-7: 31970 mM⁻¹cm⁻¹; web.expasy.org/protparam/). The protein was aliquoted, flash frozen in liquid nitrogen, and stored at −80° C. until use.

The TsOYE was produced and purified as described in Ribeaucourt et al. (ACS Catal. 12:1111-1116 (2022). Briefly: E. coli BL21(DE3) strains harboring the gene for TsOYE (grown at 37° C., 180 rpm) were induced at OD₆₀₀ of ˜0.6 with IPTG (0.1 mM, 30 min, 4° C.), resuspended in MOPS-NaOH (20 mM, pH 7.0), disrupted using a Multi Shot Cell Disruption System (two cycles) and clarified by centrifugation (17,500×g, 30 min, 4° C.). The supernatant was incubated at 70° C. for 90 min, clarified by centrifugation (38,500×g, 30 min, 4° C.), supplemented with FMN (1:1 molar ratio), incubated on ice (30 min), washed and concentrated with MOPS-NaOH (20 mM, pH 7.0) using an AMICON filter with 30 kDa cut-off. Purity was assessed by SDS-PAGE and mass spectrometry, and the enzyme was stored as aliquots at −20° C. until use.

TS-PTDH binding site conservation analysis. Sequence conservation was analyzed by performing a BLAST-P search using 5,000 max target sequences with TS-PTDH as the query to identify homologs. Hits were filtered to remove samples with low query coverage (<60%), low sequence identity (<30%), and redundant sequences (>98% sequence identity). Multiple sequence alignment with MAFFT was run, and frequencies for each of the 20 amino acids were tabulated for every non-gap column based on TS-PTDH. Sequence entropy was calculated using ProDy at each position with the formula where H is entropy in bits, pᵢ is the observed frequency for an amino acid, and the values are summed over the 20 natural residues with gaps ignored.

$$H ( X ) = - \sum \begin{matrix}20 \\ i = 1\end{matrix} {p}_{i} {log}_{2} {p}_{i} \qquad ( 1 )$$

Sequence entropy was converted to z-score and percentile rank. A final conservation score was computed by subtracting the sequence entropy percentile from 1 to rank how conserved each residue is compared to the entire population with a score of 0 indicating max variability and 1 indicating max conservation.

Five regions of interest composing the first shell of residues surrounding the NMN+ were examined based on the crystal structure of TS-PTDH with NAD⁺ bound (PDB: 4E5N). The catalytically active NMN+ binding pose is assumed to be similar to that of the native cofactor NAD⁺ as NMN⁺ fully maintains the nicotinamide ring involved in hydride transfer. These regions are: (1) the seatbelt loop from 76-79, (2) the arch which includes 97, 100, 101, and 104, (3) the Rossmann alpha helix 1 from 154-157, (4) the base loop with 207-210, and (5) the nicotinamide pocket which includes 235-237, 261-262, and 292-295.

Design of the Growth-based Selection Platform for NMN⁺-reducing Enzymes. Growth-based selections are powerful tools to alter enzyme activities and demand that the desired enzyme activity be connected to the cell's fitness. Since NAD(P)/H redox balance must be maintained for cells to survive, multiple growth-based, high-throughput selection platforms were designed to engineer NAD(P)H-dependent enzymes. Based on the same principle of coupling enzymes capable of maintaining redox balance, it was first sought to incorporate NMN(H) redox cycling into a life-essential process in E. coli, the production of reduced glutathione (GSH) (see FIG. 1A).

GSH functions to reduce undesired cysteine disulfide bonds that impair protein folding, and GSH is required for the proper function of cytosolic proteins in E. coli. A parallel and partially redundant antioxidant system is the thioredoxin (Trx) system in E. coli. When both the GSH system and the Trx system are disrupted through the genetic knockout of Gor (glutathione reductase) and TrxB (thioredoxin reductase) in the E. coli SHuffle strain, the intracellular environment becomes largely oxidative. Although the SHuffle strain is still viable under non-stressful conditions, it was postulated that it cannot combat the oxidative stress effectively, and requires a functional Gor to survive exposure to oxidants such as diamide. Thus, if Gor can be engineered to specifically utilize NMNH instead of the natural cofactors NAD(P)H, then virtually any NMN⁺-reducing enzyme can be made life-essential for E. coli by their role of supplying vital NMNH to the engineered Gor to support GSH production (see FIG. 1A).

Engineering an NMNH-specific Gor. NMN⁺ is a truncated version of the native cofactor NAD⁺ which maintains the nicotinamide ring that functions in hydride transfer and lacks the AMP recognition handle (see FIG. 1A). It was hypothesized that the ribose and phosphate groups of NMN(H) have the potential to accept new polar interactions to supplant the missing interactions from the AMP recognition handle.

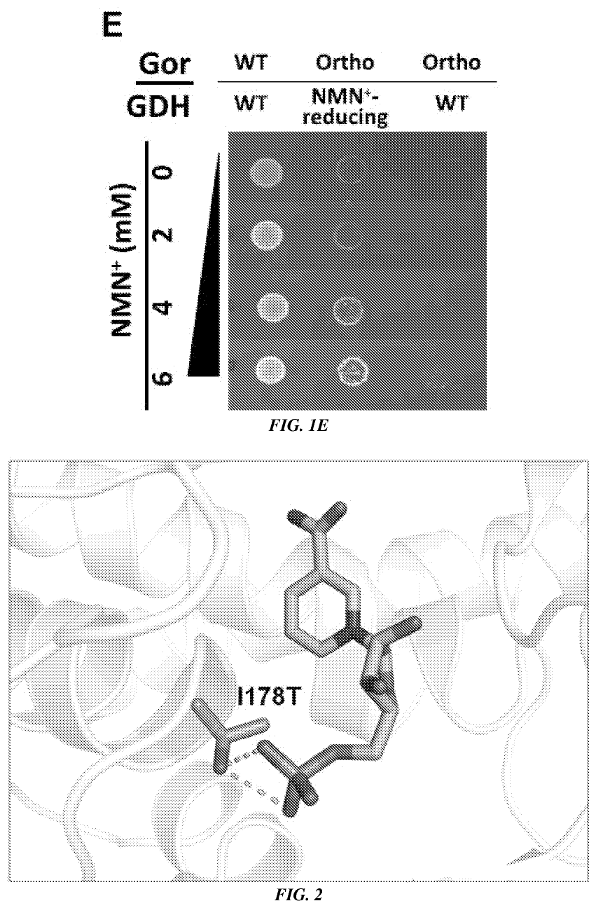

The first round of rational design on E. coli Gor (Ec Gor) aimed to create additional interactions between the protein and the free phosphate group on NMNH. Three residue sites G174, G176, and 1178 were targeted based on their proximity to the ribose-phosphate group of NMNH, and a total of eight single-mutation variants were tested (see FIGS. 1B and C). The hydroxyl group on the side chain of T178 is predicted to make two new hydrogen bonds with the phosphate group of the NMNH ligand (see FIG. 2). Indeed, I178T had 4-fold enhanced NMNH-dependent activity compared to the wild type. Another variant predicted to make favorable salt bridge interactions with the NMNH phosphate group, G174H, also showed enhanced NMNH-dependent activity (see FIGS. 1B and C). These findings reinforced the design principle of introducing novel polar contacts to the NMNH phosphate group to increase activity.

It was next sought to increase the orthogonality of Gor in using NMNH versus the natural cofactor NADPH. The wild type Ec Gor strongly prefers NADPH, and it already has low binding affinity for NADH. Furthermore, a previously identified variant with R198M-R204L double mutations had almost completely abolished NADPH-dependent activity, possibly due to ablation of interactions with the 2′-phosphate group on NADPH (see FIG. 1B). Combining the R198M-R204L double mutations on top of Ec Gor I178T caused only a modest decrease in NMNH-dependent activity (see FIGS. 1C and D). The catalytic efficiency (k_{cat}/Kₘ) for NMNH is comparable to that of the I178T, and is ˜4-fold higher than the wild type. Importantly, the triple mutant Ec Gor I178T-R198M-R204L (named Gor Ortho) has drastically reduced catalytic efficiency for NADPH, by ˜15,000-fold and ˜9,000-fold, respectively, compared to Ec Gor wild type and I178T single mutant (see FIG. 1D). The additional R198M-R204L mutations also decreased the catalytic efficiency for NADH by ˜2-fold (see FIG. 1D), making Gor Ortho equally deficient in NADH activity as the wild type.

Gor Ortho features a 60,000-fold cofactor specificity switch from its native cofactor NADPH to NMNH based on the catalytic efficiency measured in vitro (see FIG. 1D). To assess its suitability for use in the growth selection in vivo, it is important to consider the intracellular concentrations of NADH and NADPH, ˜83 μM and 120 μM, respectively, in glucose-fed exponentially growing cells. During the kinetic characterization, Gor Ortho activity could not be saturated at concentrations up to 4 mM NAD(P)H, indicating large, non-physiologically relevant Kₘ for the natural cofactors (see Table 2). Although the catalytic efficiency for NMNH is still relatively low, the intracellular concentration of NMNH can be modulated by exogenously supplementing the cofactor in the growth media, which may support Gor Ortho's NMNH-specific function in physiological conditions. Gor Ortho was then tested in the SHuffle strain for NMNH cycling-dependent growth behavior.

TABLE 2
Kinetic parameters of glutathione reductase (Gor) variants

| | Kinetic parameters | | | | | | | | |
|---|---|---|---|---|---|---|---|---|---|
| | NADH | | | NADPH | | | NMNH | | |
| | k_{cat} | Kₘ | k_{cat}/Kₘ | k_{cat} | Kₘ | k_{cat}/Kₘ | k_{cat} | Kₘ | k_{cat}/Kₘ |
| Enzyme | [s⁻¹] | [mM] | [mM⁻¹ s⁻¹] | [s⁻¹] | [mM] | [mM⁻¹ s⁻¹] | [s⁻¹] | [mM] | [mM⁻¹ s⁻¹] |
| Gor | n.d.* | n.d.* | 0.04 ± 0.01 | 84 ± 7 | 0.06 ± 0.01 | 1450 ± 119 | n.d.* | n.d.* | 0.003 ± 0.001 |
| Gor I178T | n.d.* | n.d.* | 0.09 ± 0.01 | 31 ± 1 | 0.04 ± 0.01 | 874 ± 160 | n.d.* | n.d.* | 0.014 ± 0.003 |
| Gor I178T- | | | | | | | | | |
| R198M- | n.d.* | n.d.* | 0.06 ± 0.01 | n.d.* | n.d.* | 0.10 ± 0.01 | n.d.* | n.d.* | 0.012 ± 0.001 |
| R204L | | | | | | | | | |
| (Gor | | | | | | | | | |
| Ortho) | | | | | | | | | |

Reactions were performed in 96 mM Tris-Cl buffer pH 7.5, 2.5 mM GSSG and varied cofactor concentrations. Values reported with standard deviation and a replicate size of n = 3
*Not detectable. Kₘ values are higher than 1.5 mM as the enzyme could not be saturated with the cofactor concentrations tested. For k_{cat}/Kₘ, the Michaelis-Menten equation was modified under the assumption Kₘ >> S, as previously described¹. Using the modified equation, we performed linear regression on initial reaction rate versus NMN⁺ concentration, the k_{cat}/Kₘ was determined using the resulting slope divided by enzyme concentration.

Validation of the Growth-based Selection Platform. The functional selection platform must specifically report the presence of an NMN⁺-reducing enzyme using growth as a readout. An engineered glucose dehydrogenase (GDH), Bacillus subtilis GDH Triple (I195R-A93K-Y39Q) was found to efficiently reduce NMN⁺ using glucose. When this engineered Bs GDH Triple mutant is introduced to the SHuffle strain which also harbors Gor Ortho, cell growth was observed and found to increase with greater NMN⁺ supplementation in the media (see FIG. 1E). The selection media contains 0.5 mM diamide which has been optimized to exert the appropriate level of oxidative stress. Importantly, wild type Bs GDH, which only reduces NAD(P)⁺ but not NMN⁺, did not support cell growth (see FIG. 1E). In comparison, the SHuffle strain harboring wild type Ec Gor exhibited robust growth independent of NMN⁺ supplementation, since wild type Ec Gor can rescue growth by producing GSH using intracellular NADPH (see FIG. 1E). These results demonstrate that the growth selection platform functions as intended, with the SHuffle strain carrying Gor Ortho supporting growth only with NMN⁺ cycling.

Enable Phosphite Dehydrogenase to Use NMN⁺ by Rational Design. Phosphite dehydrogenase (PTDH) carries out the NAD(P)+-dependent oxidation of phosphite (Pt) into phosphate (Pi). It has been widely applied for cofactor regeneration in industrial biocatalytic processes due to its usage of the low-cost feedstock Pt, production of the non-toxic product Pi that has additional pH buffering ability, and reaction irreversibility that can be exploited as a thermodynamic driving force.

A two-stage approach was employed with the 16× thermostable variant of Pseudomonas stutzeri PTDH (TS-PTDH) to engineer an NMN⁺-recycling PTDH: First, rational design was carried out on residue positions that directly contact NMN⁺; Second, site-saturated mutagenesis was performed targeting sites that are more remote to NMN⁺ in the cofactor binding pocket combined with high-throughput screening by the Gor Ortho growth selection platform. This two-stage workflow integrating structure-based design and semi rational mutagenesis enables the discovery of NMN+ active mutants with minimized experimental burden. The first round of design is performed to quickly acquire rationally apparent mutations that can seed exploration in the second round. Subsequently, resources are concentrated on the second-round sampling with high combinatorial variation to avoid getting trapped in local optima that pervade protein fitness landscapes.

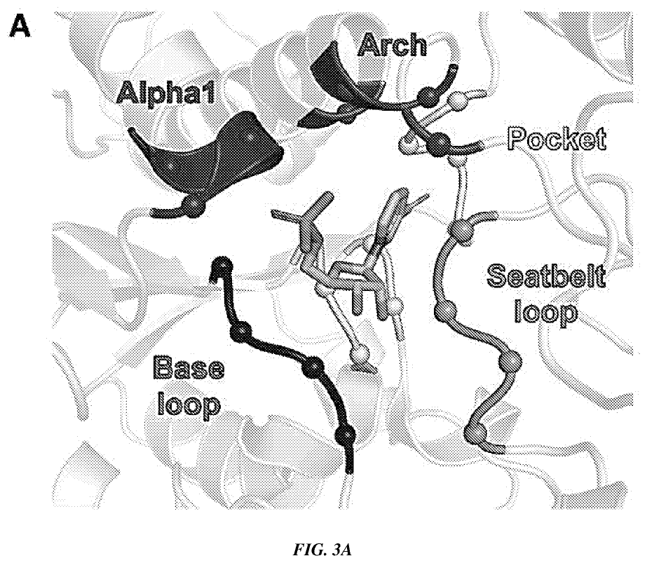

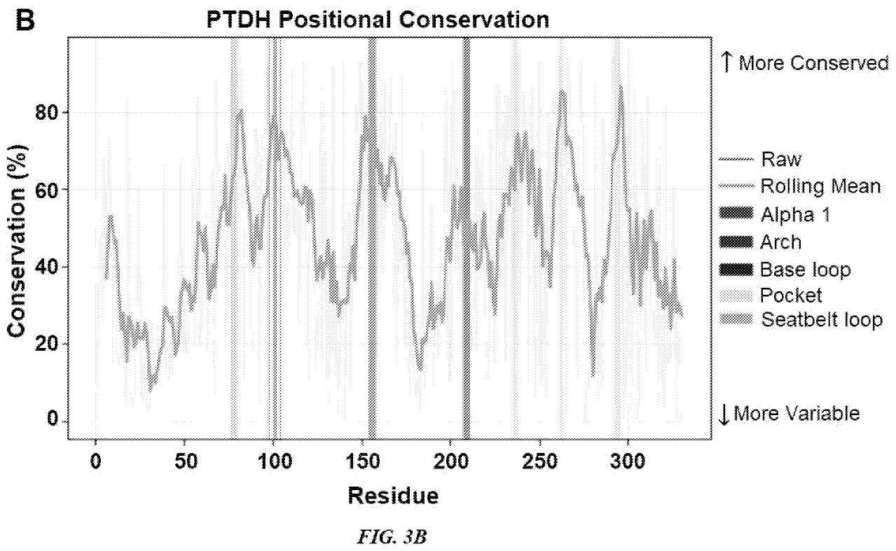

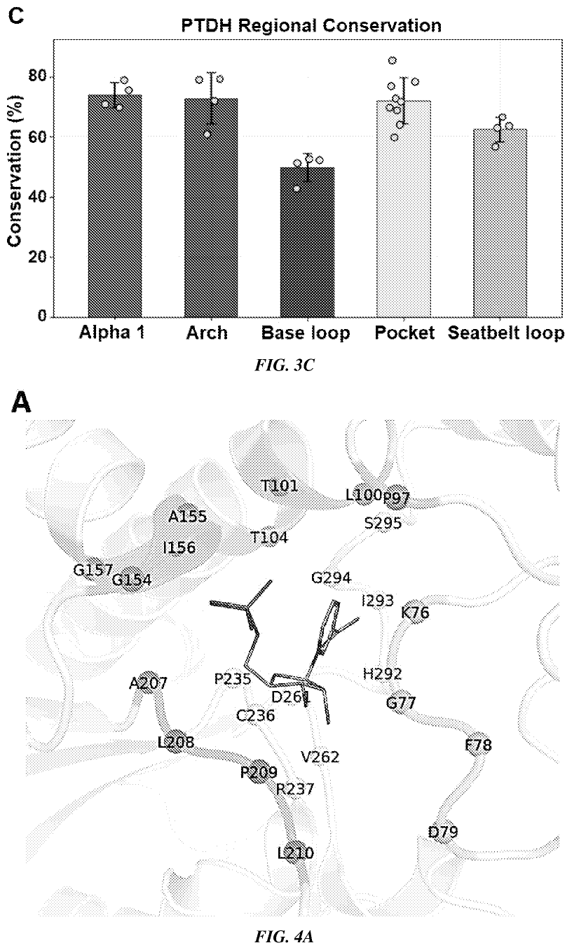

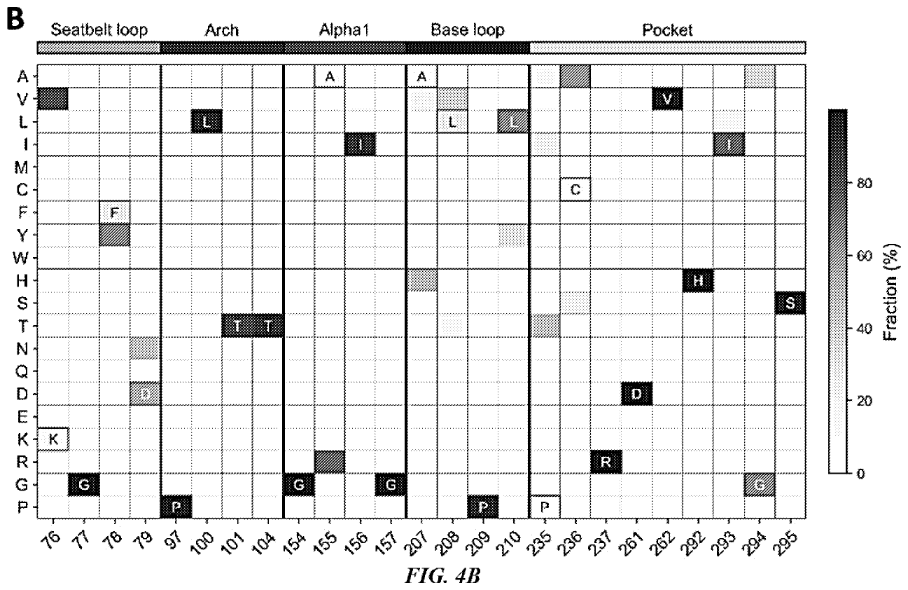

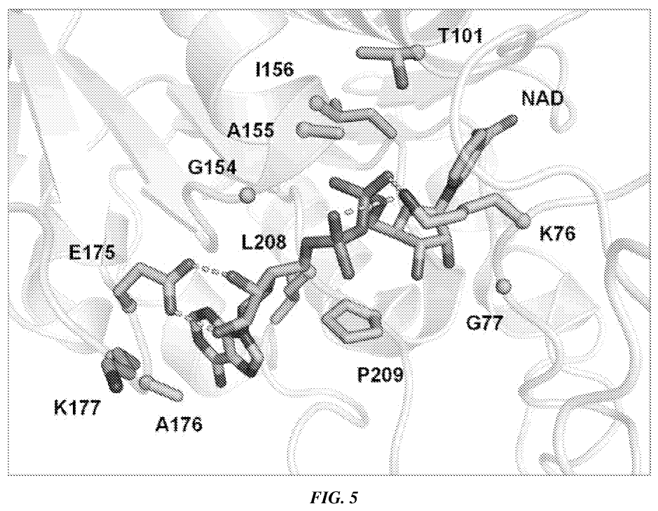

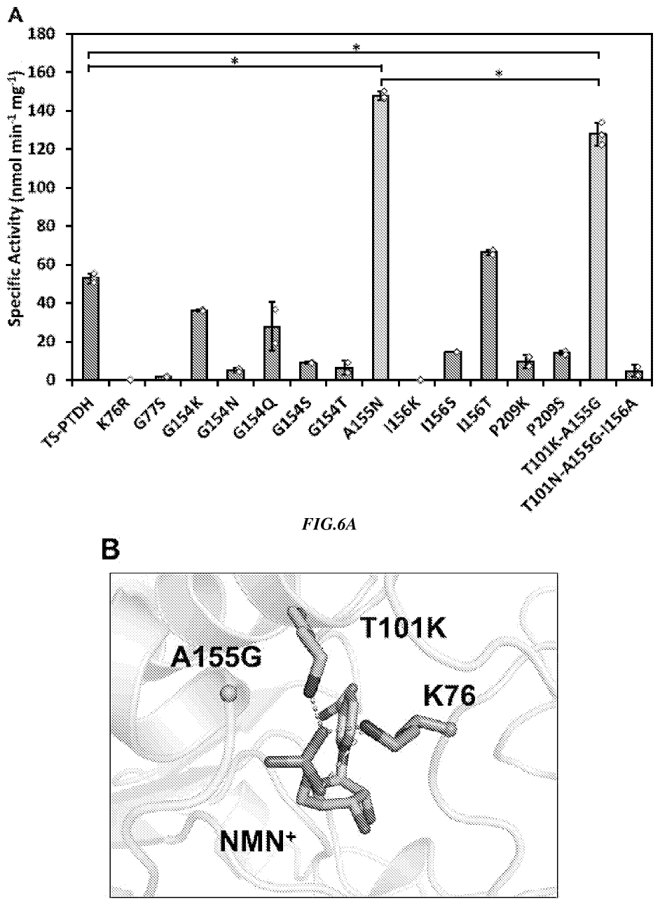

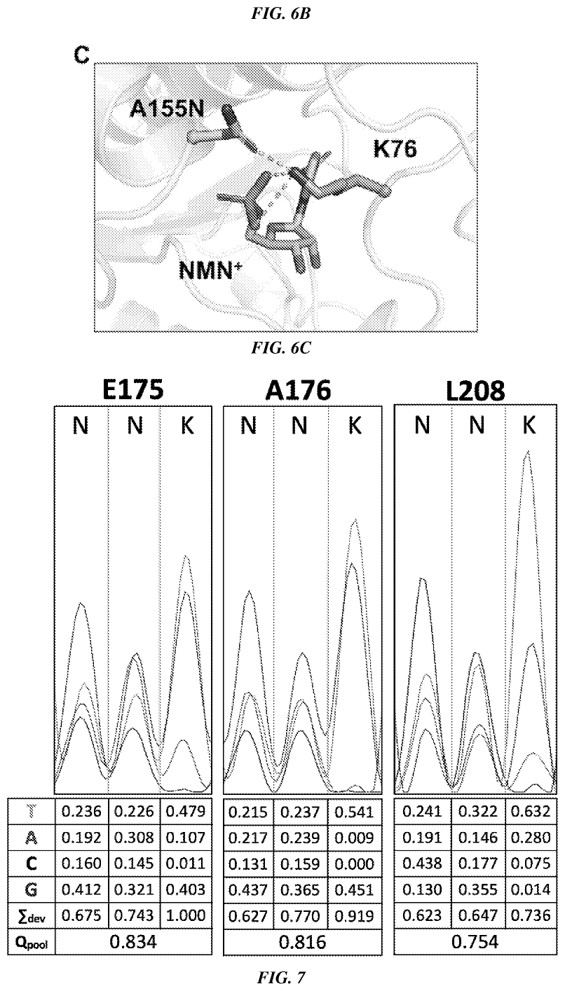

For the initial round of rational design, first shell residues were mutated to form novel polar contacts to NMN⁺, at the phosphate or ribose (see FIGS. 3 and 4). Evolutionary conservation for regions surrounding the NMN⁺ binding pocket was calculated through sequence entropy of aligned homologs to identify sites with high variability or those prone to sample polar residues (See FIGS. 3 and 4), which show that all NMN⁺ first shell residues are highly conserved. However, studies have shown that caution should be taken in equating sequence consensus to functional essentiality, especially when the desired function (i.e., NMN⁺-utilizing activity) has not been explored by evolution. Instead of bioinformatic analysis, a structure guided approach (see FIG. 5) was relied upon and was used to design fourteen variants (see FIG. 6A) to enhance NMN⁺ activity.

According to the crystal structure of TS-PTDH bound with NAD⁺, there are 11 residues lining the NAD binding pocket that can be potentially designed: K76, G77, T101, G154, A155, 1156, E175, A176, K177, L208, and P209. The positions E175, K177 and A176 were excluded as they are distal from the NMN⁺ phosphate and single mutations at these positions cannot form direct interactions with NMN⁺. Position K76 which establishes a salt-bridge with the pyrophosphate in NAD⁺ and NMN⁺ phosphate was also excluded. This side-chain polar contact is critical, validated by the variant K76R that completely lacks NMN⁺ activity (see FIG. 6A). Taken together, 6 positions (T101, A155, G154, L208, G77, and P209) were selected for the design efforts. These designable positions were mutated to polar amino acids, with preference for positively charged residues to form novel polar contacts with NMN⁺. The resulting variants were evaluated based on their shape complementarity and the possibility to form new hydrogen bonds through visual inspection. Overall, 14 variants were selected and characterized for NMN⁺ activity (see FIG. 6A).

Through specific activity assay, two variants of interest displaying enhanced NMN⁺-dependent activity, A155N and T101K-A155G were identified. The template TS-PTDH displayed NMN⁺-dependent specific activity of 53.0±3.4 nmol min⁻¹ mg⁻¹, while T101K-A155G showed roughly 2-fold, and A155N around 3-fold increase (see FIG. 6A). Further kinetic analysis of A155N indicated a ˜30-fold increase in catalytic efficiency for NMN⁺ compared to TS-PTDH (see TABLE 3).

To determine the structural basis for the improved catalysis with NMN⁺, molecular modeling was performed with Rosetta to examine potential binding modes. T101K reaches across the scaffold of the cofactor to form a salt bridge with the NMN⁺ phosphate, and A155G was necessary to clear space for T101K (see FIG. 6B). A155N extends over the NMN⁺ phosphate and forms a hydrogen bond with K76, reinforcing the existing salt bridge between K76 and the NMN⁺ phosphate (see FIG. 6C). This interaction is suggested to pre-organize K76 for cofactor binding. The variant with highest activity with NMN⁺, A155N, was further engineered.

Evolving Phosphite Dehydrogenase using High-throughput Growth Selection. Next was constructed a site-saturated mutagenesis library on top of TS-PTDH A155N targeting E175, A176, and L208. These three positions span the binding cleft surrounding the adenosine moiety of NAD⁺ and are part of the key determinants of cofactor specificity in TS-PTDH. These three sites were mutated simultaneously using NNK degenerate codons and the resultant library's quality was determined as described in Sullivan et al. (Enzym. Microb. Technol. 53:70-77 (2013)) (see FIG. 7). The resulting library was transformed into the SHuffle strain together with Gor Ortho to produce ˜2.5×10⁶ independent transformants, ensuring thorough coverage of the theoretical diversity (8×10³ variants) with 5-fold oversampling.

In the first round of selection carried out on M9 agar plates with 0.5 mM diamide, 10 g/L glucose, 10 mM sodium phosphite, 5 g/L yeast extract, and 5 mM NMN⁺ hundreds of transformants grew as isolated colonies after incubation at 37° C. for 3 days. Eight colonies were picked due to their large colony size and robust growth when re-streaked on plates with identical medium. From the 8 colonies, four PTDH variants were isolated that showed enhanced NMN⁺-dependent activity compared to the template four PTDH variants were isolated that showed enhanced NMN⁺-dependent activity compared to the template TS-PTDH A155N. Of the 4 identified variants, TS-PTDH A155N-E175Q-A176S (LY-6) and TS-PTDH A155N-E175W-A176G-L208V (LY-7) exhibited the greatest improvement, with ˜17-fold and ˜11-fold increased catalytic efficiency (k_{cat}/Kₘ) for NMN⁺ compared to the parent enzyme TS-PTDH A155N, respectively (see Table 3).

In the second round of selection, the selection pressure was increased by lowering the yeast extract concentration to 1 g/L, making the conditions even more challenging for cells to grow. Using the same growth selection process, TS-PTDH A155N-E175A-A176F (LY-13) was successfully isolated which has a catalytic efficiency for NMN⁺ ˜110-fold and ˜44-fold higher than that of TS-PTDH and TS-PTDH A155N, respectively, reaching 0.44 mM⁻¹ s⁻¹ (see Table 3). When retransformed into the growth selection strain, LY-13 demonstrated a markedly improved growth restoration relative to the A155N and wild type TSPTDH (see FIG. 8). Intriguingly, these improvements in TSPTDH activity with NMN⁺ are also shown with NADP⁺, with only a minimal decrease in activity with the preferred NAD⁺ in most cases, indicating these mutations may contribute to a global improvement in activity with a broad range of nicotinamide-based redox cofactors (see Table 3).

TABLE 3
Kinetic parameters of PTDH variants

| | Kinetic parameters | | | | | | | | |
|---|---|---|---|---|---|---|---|---|---|
| | NAD⁺ | | | NADP⁺ | | | NMN⁺ | | |
| | k_{cat} | Kₘ | k_{cat}/Kₘ | k_{cat} | Kₘ | k_{cat}/Kₘ | k_{cat} | Kₘ | k_{cat}/Kₘ |
| Enzyme | [s⁻¹] | [mM] | [mM⁻¹ s⁻¹] | [s⁻¹] | [mM] | [mM⁻¹ s⁻¹] | [s⁻¹] | [mM] | [mM⁻¹ s⁻¹] |
| TS- | 1.03 ± 0.01 | 0.06 ± 0.01 | 18 ± 1 | 0.57 ± 0.01 | 0.74 ± 0.04 | 0.77 ± 0.03 | n.d.* | n.d.* | 0.004 ± 0.001 |
| PTDH | | | | | | | | | |
| TS- | 1.46 ± 0.13 | 0.08 ± 0.01 | 18 ± 1 | 0.88 ± 0.08 | 0.63 ± 0.07 | 1.4 ± 0.1 | n.d.* | n.d.* | 0.010 ± 0.001 |
| PTDH | | | | | | | | | |
| A155N | | | | | | | | | |
| TS- | 1.04 ± 0.08 | 0.11 ± 0.01 | 9.3 ± 0.4 | 0.95 ± 0.05 | 0.05 ± 0.01 | 19 ± 1 | 0.18 ± 0.01 | 1.1 ± 0.1 | 0.17 ± 0.01 |
| PTDH | | | | | | | | | |
| A155N- | | | | | | | | | |
| E175Q- | | | | | | | | | |
| A176S | | | | | | | | | |
| (LY-6) | | | | | | | | | |
| TS- | 0.71 ± 0.04 | 0.75 ± 0.03 | 0.94 ± 0.08 | 0.56 ± 0.03 | 0.11 ± 0.01 | 5.2 ± 0.2 | 0.16 ± 0.01 | 1.4 ± 0.1 | 0.11 ± 0.01 |
| PTDH | | | | | | | | | |
| A155N | | | | | | | | | |
| E175W- | | | | | | | | | |
| A176G- | | | | | | | | | |
| L208V | | | | | | | | | |
| (LY-7) | | | | | | | | | |
| TS- | 2.06 ± 0.11 | 0.18 ± 0.03 | 12 ± 2 | 0.96 ± 0.02 | 0.05 ± 0.01 | 20 ± 1 | 0.27 ± 0.01 | 0.62 ± 0.05 | 0.44 ± 0.03 |
| A155N- | | | | | | | | | |
| E175A- | | | | | | | | | |
| A176F | | | | | | | | | |
| (LY-13) | | | | | | | | | |
| PTDH | | | | | | | | | |

Reactions were performed in 100 mM MOPS, pH 7.25, 10 mM sodium phosphite, and varied cofactor concentrations. Values reported with standard deviations and replicate size of n = 3.
*Not detectable. Kₘ values are higher than 5 mM as the enzyme could not be saturated with the cofactor concentrations tested. For k_{cat}/Kₘ, the Michaelis-Menten equation was modified under the assumption Kₘ >> S, as previously described³³. Using the modified equation, we performed linear regression on initial reaction rate versus NMN⁺ concentration, the k_{cat}/Kₘ was determined using the resulting slope divided by enzyme concentration.

Elucidating the Mechanism of Enhanced NMN⁺-dependent Activity in Engineered PTDHs. The variants identified from growth selection were further analyzed through Rosetta modeling to understand the mechanisms leading to enhanced NMN⁺-dependent activity. A common pattern observed is additional residue packing in the adenine cleft, which potentially recapitulates the role of the missing AMP when NMN⁺ is bound (see FIG. 9A).

LY-6 (TS-PTDH A155N-E175Q-A176S) is predicted to support the extension of E175Q into the space where the adenine would occupy. E175Q is able to form two hydrogen bonds, one with the backbone amide of M153 and the second with the side chain hydroxyl of A176S. These hydrogen bonds stabilize the positioning of E175Q into the binding pocket and the neighboring loops (see FIG. 9B). Both LY-7 (A155N-E175W-A176G-L208V) and LY-13 (A155N-E175A-A176F) display tight hydrophobic packing that bridges the gap between the loop emerging from the second Rossman beta strand and the loop below the bound cofactor. In LY-7, the substitution of A176G allows greater rotamer sampling for E175W, and E175W is able to twist and face L208V. L208V results in a slightly smaller residue that maintains non-polar character and packs with high shape complementarity against E175W to fill the adenine pocket (see FIG. 9C). In LY-13, the smaller E175A allows the specificity loop to move closer toward the GXGXXG glycine rich signature loop in the Rossman fold. The bulky A176F pads the void where the adenosine would fit, and it achieves minimal exposure to solvent by lying against L208 and P209 (see FIG. 9D).

Application of the engineered PTDHs in biocatalysis using noncanonical cofactors. To examine the application of the TS-PTDH variants as noncanonical redox cofactor recycling tools, an in vitro enzymatic cycling system was constructed (see FIG. 10A), coupling the TS-PTDH variants with the xenobiotic ene reductase XenA from Pseudomonas putida. This forms a closed NMN(H) cycle producing the flavor and fragrance compound levodione from ketoisophorone (KIP). First, the temporal stability of LY-13 was investigated. LY-13 was supplied at a low concentration of 0.25 μM to the reaction system, eliciting a requirement for numerous turnovers of LY-13 for the reaction to progress (see FIG. 10B). This PTDH-limited reaction system remained active for the entire 16 days it was observed, achieving a TTN of ˜45,000 with LY-13 (see FIG. 10B), a Total Turnover Frequency of ˜117 turnovers per hour averaged over the 16-day reaction. This represents the highest TTN values reported to date for a noncanonical cofactor cycling system, and it approaches the TTN values reported for the wild-type PTDH enzyme with NAD⁺ (80,000). In addition to its robust temporal stability, the low Kₘ of LY-13 with NMN⁺ (see Table 3) has been shown to be advantageous. When the cycling system utilizing LY-13 was directly compared with a system using GDH Triple, a previously reported NMNH recycling protein (Kₘ=6.4 mM, k_{cat}=3.1 s⁻¹), both systems completely converted the 33 mM KIP pool in 12 hours when 5 mM of NMN⁺ was supplied (see FIG. 10C). However, when the NMN⁺ supplementation was decreased to 1 mM and lower, the LY-13 enabled higher biotransformation rates and final titers compared to GDH Triple (see FIG. 10C).

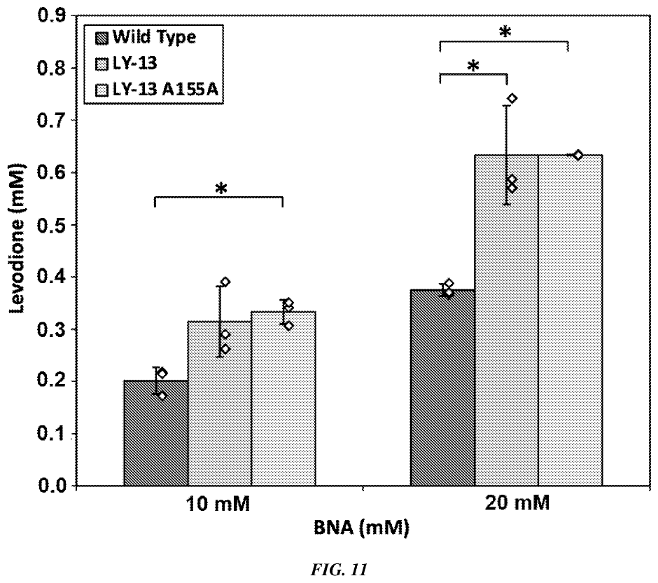

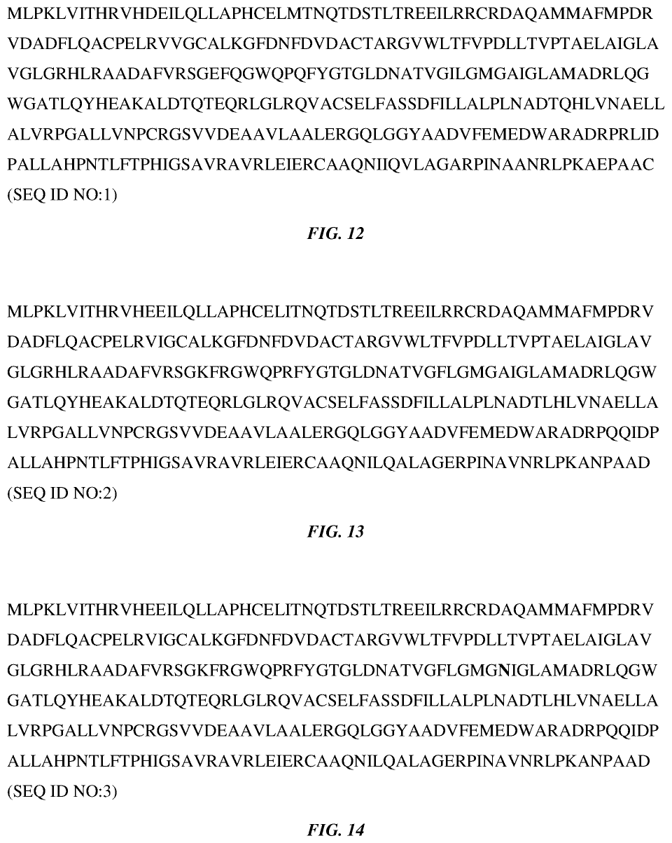

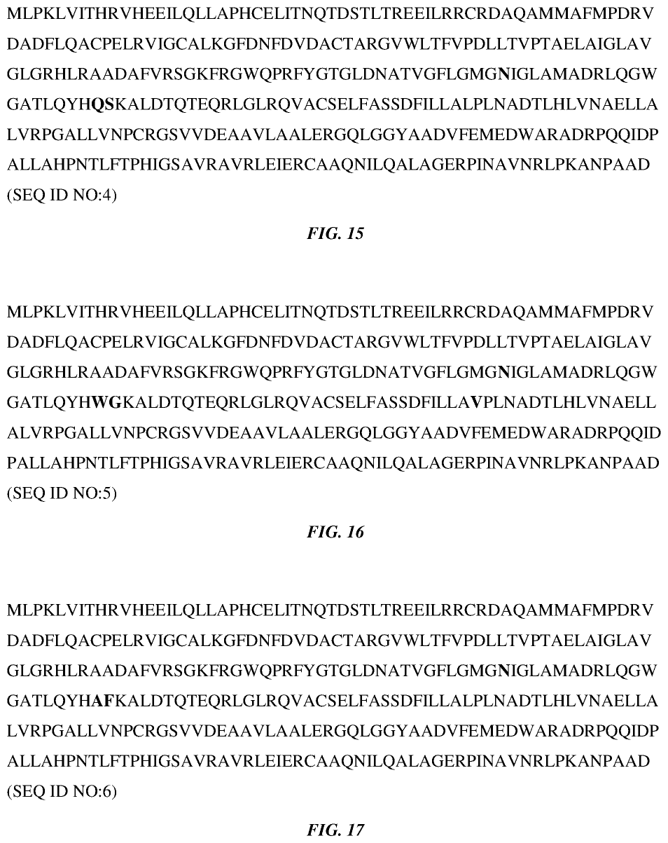

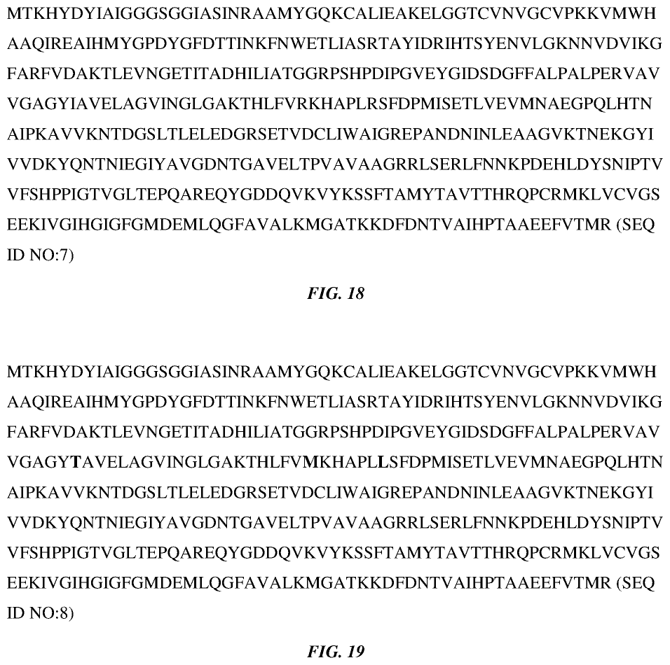

With the perspective of industrial scale, the cost of the recycled cofactor used can be decreased by switching NMN⁺ for a more cost-effective cofactor. Cofactor versatility of the PTDH variants was further explored, by exchanging NMN⁺ for an inexpensive, synthetic cofactor, BNA⁺ in the biotransformation system. In addition, XenA was exchanged with another ene reductase from Thermus scotoductus, TsOYE, which has been reported to exhibit improved activity with the reduced form of BNA⁺, BNAH. When BNA⁺ was reduced with wild type TS-PTDH, LY-7, and LY-13, a cofactor dose-dependent response in biotransformation efficiency was observed, and at 20 mM BNA⁺ supplementation, LY-7 and LY-13 showed improved utilization of the BNA⁺ pool relative to the wild type protein (see FIG. 10D). Interestingly, the relative increase in BNA(H)-dependent biotransformation efficiency was consistent with the improvements in NMN⁺ catalytic efficiency (see Table 3), regardless of, albeit much lower in absolute terms due to the drastic structural differences between these two noncanonical cofactors (see FIG. 10D). This supports the hypothesis that the adenine-cleft filling mutations, regardless of the NMN⁺-hydrogen binding mutation (see FIG. 11), are responsible for more global changes in the protein which enhanced acceptance of noncanonical cofactors in general.

Certain embodiments of the invention have been described. It will be understood that various modifications may be made without departing from the spirit and scope of the invention. Other embodiments are within the scope of the following claims.

What is claimed is:

1. A recombinantly engineered polypeptide that has improved catalytic efficiency for an oxidized form of a noncanonical cofactor, wherein the recombinantly engineered polypeptide comprises from 1 to 12 amino acid mutations in comparison to the sequence of the wild-type or parent polypeptide, or a thermostable variant thereof;
wherein the amino acid mutations promote catalytic efficiency of the recombinantly engineered polypeptide in reducing the oxidized form of the noncanonical cofactor and/or disrupt electrostatic complementarity between the recombinantly engineered polypeptide and a natural cofactor, and wherein a polypeptide having the sequence of the wild-type or parent polypeptide, or a thermostable variant thereof, has minimal to very limited catalytic efficiency for the oxidized form of the noncanonical cofactor;
wherein the recombinantly engineered polypeptide is configured to in vivo recycle the nonconical cofactor when used with a second recombinantly engineered polypeptide that utilizes the reduced form of noncanonical cofactor for cell growth.

2. The recombinantly engineered polypeptide of claim 1, wherein the recombinantly engineered polypeptide has at least 100-fold catalytic efficiency for the oxidized form of the noncanonical cofactor in comparison to the wild-type or parent polypeptide, or a thermostable variant thereof.

3. The recombinantly engineered polypeptide of claim 1, wherein the noncanonical cofactor is selected from the group consisting of nicotinamide mononucleotide (NMN), 1-phenyl-1,4,-dihydronicotinamide (PNA), 1-benzyl-1,4-dihydronicotinamide (BNA), 1-(4-hydroxyphenyl)1,4-dihydronicotinamide (HPNA), 1-methyl-1,4-dihydronicotinamide (MNA), nicotinamide flucytosine dinucleotide (NFCD), nicotinamide mononucleoside (NR), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile.

4. The recombinantly engineered polypeptide of claim 1, wherein the wild-type or parent polypeptide, or a thermostable variant thereof encodes a dehydrogenase or an oxidoreductase selected from phosphite dehydrogenase (PTDH), alcohol dehydrogenase (NAD), alcohol dehydrogenase (NADP), glutathione reductase, homoserine dehydrogenase, glucose dehydrogenase, glycerol dehydrogenase, propanediol-phosphate dehydrogenase, glycerol-3-phosphate dehydrogenase (NAD⁺), lactate dehydrogenase, malate dehydrogenase, isocitrate dehydrogenase, acetaldehyde dehydrogenase, glyceraldehyde 3-phosphate dehydrogenase, pyruvate dehydrogenase, oxoglutarate dehydrogenase, and formate dehydrogenase, or a thermostable variant of any one of the foregoing.

5. The recombinantly engineered polypeptide of claim 1, wherein the recombinantly engineered polypeptide comprises a sequence that is at least 98% identical to the sequence of SEQ ID NO:3, SEQ ID NO:4, SEQ ID NO:5, or SEQ ID NO:6, and has PTDH activity.

6. A recombinantly engineered polypeptide that has improved catalytic efficiency for a reduced form of a noncanonical cofactor,
wherein the recombinantly engineered polypeptide comprises from 3 to 12 amino acid mutations in comparison to the sequence of the wild-type or parent polypeptide, or a thermostable variant thereof;
wherein the amino acid mutations promote catalytic efficiency of the recombinantly engineered polypeptide for the reduced form of the noncanonical cofactor and/or reduces catalytic activity for natural cofactor(s), and
wherein a polypeptide having the sequence of the wild-type or parent polypeptide, or a thermostable variant thereof, has minimal to very limited catalytic efficiency for the reduced form of the noncanonical cofactor; wherein the recombinantly engineered polypeptide uses the reduced noncanonical cofactor for cell growth.

7. The recombinantly engineered polypeptide of claim 6, wherein the recombinantly engineered polypeptide has at least 4-fold catalytic efficiency for the reduced form of the noncanonical cofactor in comparison to the wild-type or parent polypeptide, or a thermostable variant thereof.

8. The recombinantly engineered polypeptide of claim 6, wherein the noncanonical cofactor is selected from the group consisting of nicotinamide mononucleotide (NMN), 1-phenyl-1,4,-dihydronicotinamide (PNA), 1-benzyl-1,4-dihydronicotinamide (BNA), 1-(4-hydroxyphenyl)1,4-dihydronicotinamide (HPNA), 1-methyl-1,4-dihydronicotinamide (MNA), nicotinamide flucytosine dinucleotide (NFCD), nicotinamide mononucleoside (NR), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile.

9. The recombinantly engineered polypeptide of claim 6, wherein the wild-type or parent polypeptide, or a thermostable variant thereof, has the sequence of SEQ ID NO:7.

10. The recombinantly engineered polypeptide of claim 6, wherein the recombinantly engineered polypeptide comprises a sequence that is at least 98% identical to the sequence of SEQ ID NO:8, and has glutathione reductase activity.

11. A growth selection process to identify recombinantly engineered polypeptides capable of cycling an oxidized form of a noncanonical cofactor, the growth selection process comprising:
expressing in microorganisms:
(i) a first recombinantly engineered polypeptide that comprises from 3 to 12 amino acid mutations in comparison to the sequence of the wild-type or parent polypeptide, or a thermostable variant thereof, and which can utilize a reduced form of a noncanonical cofactor to promote microorganism growth; and
(ii) a second recombinantly engineered polypeptide that comprises from 1 to 12 amino acid mutations in comparison to the sequence of the wild-type or parent polypeptide, or a thermostable variant thereof, wherein the introduced amino acid mutations are designed to increase the activity of the recombinantly engineered polypeptide to reduce an oxidized form of the noncanonical cofactor;
culturing the microorganisms using a first selection criteria to identify variants with enhanced noncanonical-dependent activity, wherein the first selection criteria select for variants that show enhanced noncanonical cofactor activity by exhibiting an increased growth or growth rate in comparison to other transformed microorganisms;
optionally, culturing the variants using additional selection criteria, wherein the additional selection criteria make it more, and more, challenging for the microorganisms to grow without having catalytic efficiency for the noncanonical cofactor.

12. The growth selection process of claim 11, wherein the microorganisms are engineered to have an intracellular environment that is largely oxidative, by the disruption of gene(s) that encode protein(s) which reduce intracellular oxidative stress.

13. The growth selection process of claim 12, wherein the gene(s) for glutathione reductase (Gor) and/or thioredoxin reductase (TrxB) are disrupted.

14. The growth selection process of claim 13, wherein the first recombinantly engineered polypeptide comprises a sequence that is at least 98% identical to the sequence of SEQ ID NO:8, and has glutathione reductase activity.

15. The growth selection process of claim 11, wherein the second recombinantly engineered polypeptide comprises a sequence that is at least 85% identical to a sequence from a dehydrogenase or from an oxidoreductase.

16. The growth selection process of claim 15, wherein the dehydrogenase or the oxidoreductase is selected from phosphite dehydrogenase (PTDH), alcohol dehydrogenase (NAD), alcohol dehydrogenase (NADP), glutathione reductase, homoserine dehydrogenase, glucose dehydrogenase, glycerol dehydrogenase, propanediol-phosphate dehydrogenase, glycerol-3-phosphate dehydrogenase (NAD⁺), lactate dehydrogenase, malate dehydrogenase, isocitrate dehydrogenase, acetaldehyde dehydrogenase, glyceraldehyde 3-phosphate dehydrogenase, pyruvate dehydrogenase, oxoglutarate dehydrogenase, and formate dehydrogenase.

17. The growth selection process of claim 11, wherein the noncanonical cofactor is selected from the group consisting of nicotinamide mononucleotide (NMN), 1-phenyl-1,4,-dihydronicotinamide (PNA), 1-benzyl-1,4-dihydronicotinamide (BNA), 1-(4-hydroxyphenyl)1,4-dihydronicotinamide (HPNA), 1-methyl-1,4-dihydronicotinamide (MNA), nicotinamide flucytosine dinucleotide (NFCD), nicotinamide mononucleoside (NR), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile.

18. The growth selection process of claim 11, wherein the amino acid mutations are selected by using a protein modeling program that predicts a protein structure de novo.

19. The growth selection process of claim 11, wherein the first selection criteria include providing culture conditions comprising the noncanonical cofactor and a thiol oxidizing agent, and selecting for variants that exhibit the greatest growth and/or growth rates.

20. The growth selection process of claim 11, wherein the additional selection criteria include providing culture conditions of the first selection criteria but with a reduced concentration of a feedstock, and selecting for variants that exhibit the greatest growth and/or growth rates.


SEQUENCE LISTING (8 sequences)

SEQ ID NO: 1 - protein, 336 aa, Pseudomonas stutzeri
MLPKLVITHRVHDEILQLLAPHCELMTNQTDSTLTREEILRRCRDAQAMMAFMPDRVDADFLQACPELRVVGCALKGFDNFDVDACTARGVWLTFVPDLLTVPTAELAIGLAVGLGRHLRAADAFVRSGEFQGWQPQFYGTGLDNATVGILGMGAIGLAMADRLQGWGATLQYHEAKALDTQTEQRLGLRQVACSELFASSDFILLALPLNADTQHLVNAELLALVRPGALLVNPCRGSVVDEAAVLAALERGQLGGYAADVFEMEDWARADRPRLIDPALLAHPNTLFTPHIGSAVRAVRLEIERCAAQNIIQVLAGARPINAANRLPKAEPAAC

SEQ ID NO: 2 - protein, 336 aa, Pseudomonas stutzeri
MLPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPRFYGTGLDNATVGFLGMGAIGLAMADRLQGWGATLQYHEAKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKANPAAD

SEQ ID NO: 3 - protein, 336 aa, synthetic construct
MLPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPRFYGTGLDNATVGFLGMGNIGLAMADRLQGWGATLQYHEAKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKANPAAD

SEQ ID NO: 4 - protein, 336 aa, synthetic construct
MLPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPRFYGTGLDNATVGFLGMGNIGLAMADRLQGWGATLQYHQSKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKANPAAD

SEQ ID NO: 5 - protein, 336 aa, synthetic construct
MLPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPRFYGTGLDNATVGFLGMGNIGLAMADRLQGWGATLQYHWGKALDTQTEQRLGLRQVACSELFASSDFILLAVPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKANPAAD

SEQ ID NO: 6 - protein, 336 aa, synthetic construct
MLPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPRFYGTGLDNATVGFLGMGNIGLAMADRLQGWGATLQYHAFKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKANPAAD

SEQ ID NO: 7 - protein, 450 aa, Escherichia coli
MTKHYDYIAIGGGSGGIASINRAAMYGQKCALIEAKELGGTCVNVGCVPKKVMWHAAQIREAIHMYGPDYGFDTTINKFNWETLIASRTAYIDRIHTSYENVLGKNNVDVIKGFARFVDAKTLEVNGETITADHILIATGGRPSHPDIPGVEYGIDSDGFFALPALPERVAVVGAGYIAVELAGVINGLGAKTHLFVRKHAPLRSFDPMISETLVEVMNAEGPQLHTNAIPKAVVKNTDGSLTLELEDGRSETVDCLIWAIGREPANDNINLEAAGVKTNEKGYIVVDKYQNTNIEGIYAVGDNTGAVELTPVAVAAGRRLSERLFNNKPDEHLDYSNIPTVVFSHPPIGTVGLTEPQAREQYGDDQVKVYKSSFTAMYTAVTTHRQPCRMKLVCVGSEEKIVGIHGIGFGMDEMLQGFAVALKMGATKKDFDNTVAIHPTAAEEFVTMR

SEQ ID NO: 8 - protein, 450 aa, synthetic construct
MTKHYDYIAIGGGSGGIASINRAAMYGQKCALIEAKELGGTCVNVGCVPKKVMWHAAQIREAIHMYGPDYGFDTTINKFNWETLIASRTAYIDRIHTSYENVLGKNNVDVIKGFARFVDAKTLEVNGETITADHILIATGGRPSHPDIPGVEYGIDSDGFFALPALPERVAVVGAGYTAVELAGVINGLGAKTHLFVMKHAPLLSFDPMISETLVEVMNAEGPQLHTNAIPKAVVKNTDGSLTLELEDGRSETVDCLIWAIGREPANDNINLEAAGVKTNEKGYIVVDKYQNTNIEGIYAVGDNTGAVELTPVAVAAGRRLSERLFNNKPDEHLDYSNIPTVVFSHPPIGTVGLTEPQAREQYGDDQVKVYKSSFTAMYTAVTTHRQPCRMKLVCVGSEEKIVGIHGIGFGMDEMLQGFAVALKMGATKKDFDNTVAIHPTAAEEFVTMR